(19) United States
(12) Patent Application Publication
(10) Pub. No.: US 2025/0204936 A1
(43) Pub. Date: Jun. 26, 2025

(54) CATHETER TIP FOR ASPIRATION SYSTEM

(71) Applicant: Walk Vascular, LLC, Irvine, CA (US)

(72) Inventors: Randolf von Oepen, Aptos, CA (US); Adam K. Hoopai, Highland Park, IL (US); Ted Su, Sunnyvale, CA (US); Jared Ryan G. Lumauig, San Jose, CA (US); Rafael A. Castellanos Alvarez, San Jose, CA (US)

(21) Appl. No.: 18/965,087

(22) Filed: Dec. 2, 2024

Related U.S. Application Data
(60) Provisional application No. 63/613,971, filed on Dec. 22, 2023.

(51) Int. Cl. A61B 17/22; A61B 17/3203; A61M 25/00

(52) U.S. Cl. CPC A61B 17/22; A61B 17/32037; A61M 25/0068; A61B 2017/22079

(57) ABSTRACT

Disclosed are embodiments of an aspiration catheter comprising a tissue encroachment prevent assembly for preventing vessel tissue from being drawn into the aspiration lumen and/or into close proximity to an injection of pressurized fluid from a supply lumen during aspiration of a thrombus.

### CROSS-REFERENCE TO RELATED APPLICATIONS

This application claims priority to and the benefit of U.S. Provisional Patent Application Ser. No. 63/613,971, filed Dec. 22, 2023, and entitled “CATHETER TIP FOR ASPIRATION SYSTEM,” the disclosure of which is incorporated herein by this reference.

### BACKGROUND

### Technical Field

The present disclosure pertains generally to medical devices and methods of their use. More particularly, the present invention pertains to aspiration and thrombectomy devices and methods of use thereof.

### Description of the Related Art

Several devices and systems already exist to aid in the removal of thrombotic material. These include simple aspiration tube type devices using vacuum syringes to extract thrombus into the syringe, simple flush-and-aspirate devices, more complex devices with rotating components that pull in, macerate and transport thrombotic material away from the distal tip using a mechanical auger, and systems that use very high pressure fluid to macerate the thrombus and create a venturi effect to flush the macerated material away.

All of the devices described above have limitations as a result of individual design characteristics. For example, simple aspiration catheters offer ease of use and rapid deployment but may become blocked or otherwise inoperable when faced with older, more organized thrombotic material. Such devices must be removed and cleared outside the body and then re-inserted into the vasculature, which lengthens the time needed for the procedure and increases the opportunity to kink the catheter shaft. Such kinks may reduce performance by decreasing the cross-sectional area of the catheter or may render the device inoperable.

Mechanical rotary devices use an auger to grab and carry the thrombus away from the target area. Some create transport force via vacuum bottles while others create differential pressure at the distal tip of the device with the auger acting as a low-pressure pump. These devices typically work slowly and offer the physician no feedback as to when the device should be advanced further into the lesion.

Flushing type devices include manual flush type devices in which the physician manipulates a hand-driven pump to provide flowing saline at the tip of the device to break up and aspirate the thrombus material, which may introduce performance variations based on the ability of the physician to consistently pump the device over the duration of the procedure.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Embodiments of the present invention provide systems, methods, and devices for aspirating thrombus or material within a patient's body.

Implementations of the present invention solve one or more problems in the art with systems, methods, and devices for aspirating thrombus or material within a patient's body. For instance, the present invention relates to aspiration catheters that prevent vessel tissue from being drawn into an aspiration lumen of the aspiration catheter and coming into close proximity to an orifice for jetted or pressurized fluid from one or more supply lumens during aspiration; a system for aspirating thrombus, comprising an aspiration catheter including an elongate shaft configured for placement within a blood vessel of a subject, one or more supply lumens and an aspiration lumen each extending along the shaft, and an opening (or “orifice”) at or near the distal end of the one or more supply lumen, the opening configured to allow the injection of pressurized fluid into the aspiration lumen at or near the distal end of the aspiration lumen when the pressurized fluid is caused or allowed to flow through the one or more supply lumen; a tubing set comprising a first conduit having a distal end configured to couple to the aspiration lumen of the aspiration catheter and a proximal end configured to couple to a vacuum source, and a second conduit having a distal end configured to couple to the one or more supply lumen of the aspiration catheter and a proximal end configured to couple to a first fluid source; a pressurization element configured to couple to the tubing set and further configured to pressurize fluid from the first fluid source or allow pressurized fluid from the first fluid source to be transferred to the one or more supply lumens, such that the pressurized fluid is capable of flowing through the one or more supply lumens from the proximal end of the supply lumen to the distal end of the supply lumen; and wherein the vacuum source comprises a vacuum canister having two or more ports, including a first port configured to couple to a proximal end of the first conduit of the tubing set, and wherein the vacuum canister is configured to separate thrombus from aspirant delivered via the first conduit of the tubing set.

In still another configuration an aspiration catheter of this disclosure includes an elongate shaft configured for placement within a blood vessel, a supply lumen and an aspiration lumen each extending along the shaft, the supply lumen having a proximal end and a distal end, and the aspiration lumen having a proximal end and a distal opening, an orifice at or near the distal end of the supply lumen, the orifice being configured to allow injection of pressurized fluid into the aspiration lumen at or near the distal end of the aspiration lumen when the pressurized fluid is delivered through the supply lumen, and a tissue encroachment prevention assembly for preventing the blood vessel from being drawn into said aspiration lumen and coming into close proximity to the orifice of the supply lumen during aspiration.

In still another configuration an aspiration catheter of this disclosure includes an elongate shaft configured for placement within a blood vessel, the elongate shaft having a distal end with an opening at the distal end, a supply lumen and an aspiration lumen each extending along the shaft, the supply lumen having a proximal end and a distal end, and the aspiration lumen having a proximal end and a distal opening, an orifice near the distal end of the supply lumen, the orifice being configured to allow injection of pressurized fluid into the aspiration lumen at or near the distal end of the aspiration lumen when the pressurized fluid is delivered through the supply lumen, and a means for preventing the blood vessel from being drawn into the aspiration lumen and into close proximity to the orifice of the supply lumen during aspiration.

In still another configuration an aspiration catheter of this disclosure includes an elongate shaft configured for placement within a blood vessel, the elongate shaft having a distal end with an opening at the distal end, a supply lumen and an aspiration lumen each extending along an interior of the shaft, coextensive with the shaft, the supply lumen having a proximal end and a distal end, and the aspiration lumen having a proximal end and a distal opening, an orifice near the distal end of the supply lumen, the orifice being configured to allow injection of pressurized fluid into the aspiration lumen at or near the distal end of the aspiration lumen when the pressurized fluid is pumped through the supply lumen, and a tissue encroachment prevention assembly for preventing the blood vessel from being drawn into the aspiration lumen and into close proximity to the orifice of the supply lumen during aspiration, the tissue encroachment prevention assembly comprising at least one of a cage-like structure.

In still another configuration, system of this disclosure includes an aspiration catheter comprising an elongate shaft configured for placement within a blood vessel of a subject, a supply lumen and an aspiration lumen each extending along an interior of the elongate shaft, and an orifice at or near a distal end of the supply lumen, the orifice being configured to allow injection of pressurized fluid into the aspiration lumen at or near the distal end of the aspiration lumen when the pressurized fluid is caused or allowed to flow through the supply lumen. The system also can include a tubing set comprising a first conduit having a distal end configured to couple to the aspiration lumen of the aspiration catheter and a proximal end configured to couple to a vacuum source, and a second conduit having a distal end configured to couple to the supply lumen of the aspiration catheter and a proximal end configured to couple to a first fluid source, a pressurization element configured to couple to the tubing set and further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen, a means for preventing the blood vessel from being drawn into the aspiration lumen and into close proximity to the orifice of the supply lumen during aspiration.

In still another configuration, system of this disclosure includes an aspiration catheter comprising an elongate shaft configured for placement within a blood vessel of a subject, a supply lumen and an aspiration lumen each extending along an interior of the elongate shaft, and an orifice at or near a distal end of the supply lumen, the orifice being configured to allow injection of pressurized fluid into the aspiration lumen at or near the distal end of the aspiration lumen when the pressurized fluid is caused or allowed to flow through the supply lumen. The system also can include a tubing set comprising a first conduit having a distal end configured to couple to the aspiration lumen of the aspiration catheter and a proximal end configured to couple to a vacuum source, and a second conduit having a distal end configured to couple to the supply lumen of the aspiration catheter and a proximal end configured to couple to a first fluid source, a pressurization element configured to couple to the tubing set and further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen, and a tissue encroachment prevention assembly for preventing the blood vessel from being drawn into the aspiration lumen and into close proximity to the orifice of the supply lumen during aspiration.

In still another configuration, an aspiration catheter of this disclosure includes an elongate shaft configured for placement within a blood vessel, a supply lumen and an aspiration lumen each extending along an interior of the shaft, coextensive with the shaft, the supply lumen having a proximal end and a distal end, and the aspiration lumen having a proximal end and a distal opening, an orifice at or near the distal end of the supply lumen, the orifice configured to allow injection of pressurized fluid into the aspiration lumen at or near the distal end of the aspiration lumen when the pressurized fluid is pumped through the supply lumen, and a means for preventing the blood vessel from being drawn into said aspiration lumen and coming into close proximity to the orifice of the supply lumen during aspiration.

In still another configuration, an aspiration catheter includes: an elongate shaft configured for placement within a blood vessel, the elongate shaft having an aspiration lumen extending therethrough, the aspiration lumen having an opening at a distal end of the elongate shaft; a supply lumen extending along the shaft, the supply lumen having a distal end and an orifice at or near the distal end, the orifice being configured to allow injection of pressurized fluid into the aspiration lumen at or near a distal end of the aspiration lumen when the pressurized fluid is delivered through the supply lumen; and a tissue encroachment prevention assembly for preventing the blood vessel from being drawn into the aspiration lumen and coming into close proximity to the orifice of the supply lumen during aspiration, the tissue encroachment prevention assembly comprising one or more atraumatic members that expend distally from the elongate shaft and across the opening to the aspiration lumen.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 is a plan view of exemplary disposable components of a system for aspirating thrombus according to an implementation of the present disclosure.

FIG. 2 is a sectional view of an exemplary distal end of the aspiration catheter of the system for aspirating thrombus of FIG. 1 according to an implementation of the present disclosure.

FIG. 3 is a detail view of an exemplary y-connector of the aspiration catheter of the system for aspirating thrombus of FIG. 1 according to an implementation of the present disclosure.

FIG. 4 is a plan view of exemplary disposable components of a system for aspirating thrombus according to an implementation of the present disclosure.

FIG. 5 is a perspective view of an exemplary system for aspirating thrombus of FIG. 3 according to an implementation of the present disclosure.

FIG. 6 is a perspective view of an exemplary aspiration catheter according to an implementation of the present disclosure.

FIG. 7 is a sectional view of an exemplary aspiration catheter according to an implementation of the present disclosure.

FIG. 8 illustrates the exemplary aspiration catheter within a body lumen aspirating thrombus according to an implementation of the present disclosure.

FIG. 9a is a perspective view of an exemplary aspiration catheter comprising a tissue encroachment prevention assembly according to an implementation of the present disclosure.

FIG. 9b is a perspective view of the tissue encroachment prevention assembly of the exemplary aspiration catheter of FIG. 9a according to an implementation of the present disclosure.

FIG. 9c is a bottom view of the tissue encroachment prevention assembly of the exemplary aspiration catheter of FIGS. 9a and 9b according to an implementation of the present disclosure.

FIG. 9d is a flat view tissue encroachment prevention assembly in a flat view of the exemplary aspiration catheter of FIGS. 9a and 9b according to an implementation of the present disclosure.

FIGS. 10a-10b illustrate use of the exemplary aspiration catheter within a body lumen aspirating thrombus, with the tissue encroachment prevention assembly preventing tissue damage according to an implementation of the present disclosure.

FIG. 11 is a perspective view of an exemplary aspiration catheter comprising an alternate tissue encroachment prevention assembly according to an implementation of the present disclosure.

FIG. 12a is a top view of another tissue encroachment prevention assembly an exemplary aspiration catheter according to an implementation of the present disclosure.

FIG. 12b is another tissue encroachment prevention assembly in a flat view of the exemplary aspiration catheter of FIG. 12a according to an implementation of the present disclosure.

FIG. 13a is a perspective view of an exemplary aspiration catheter comprising another tissue encroachment prevention assembly according to an implementation of the present disclosure.

FIG. 13b is a cross-sectional view of the exemplary aspiration catheter of FIG. 13a comprising another tissue encroachment prevention assembly according to an implementation of the present disclosure.

FIG. 13c is a bottom view of a portion of the another tissue encroachment prevention assembly of the exemplary aspiration catheter of FIGS. 13a and 13b according to an implementation of the present disclosure.

FIG. 13d is a flat view of the another tissue encroachment prevention assembly in a flat view of the exemplary aspiration catheter of FIGS. 13a and 13b according to an implementation of the present disclosure.

FIG. 14 is a perspective view of another alternate tissue encroachment prevention assembly according to an implementation of the present disclosure.

FIG. 15 is a perspective view of another alternate tissue encroachment prevention assembly according to an implementation of the present disclosure.

FIG. 16 is a perspective view of another alternate tissue encroachment prevention assembly according to an implementation of the present disclosure.

FIG. 17 is a perspective view of another alternate tissue encroachment prevention assembly according to an implementation of the present disclosure.

FIG. 18 is a perspective view of another alternate tissue encroachment prevention assembly according to an implementation of the present disclosure.

FIGS. 19a-19b illustrates a cross-sectional side view of an alternate exemplary aspiration catheter usable to advance a tissue encroachment prevention assembly into thrombus according to an implementation of the present disclosure.

FIG. 20a-20b illustrates the alternate exemplary aspiration catheter of FIGS. 19a-19b in use within a body lumen aspirating thrombus, with the tissue encroachment prevention assembly being advance through thrombus according to an implementation of the present disclosure.

FIG. 21 illustrates a cross-sectional side view of another alternate exemplary aspiration catheter usable to advance a tissue encroachment prevention assembly into thrombus according to an implementation of the present disclosure.

FIG. 22 illustrates a cross-sectional side view of another alternate exemplary aspiration catheter usable to advance a tissue encroachment prevention assembly into thrombus according to an implementation of the present disclosure.

FIG. 23 is a perspective view of an exemplary aspiration catheter comprising another tissue encroachment prevention assembly according to an implementation of the present disclosure.

FIG. 24a is a cross-sectional view of the exemplary aspiration catheter of FIG. 23 with a tissue encroachment prevention assembly in a pre-deployed configuration or state according to an implementation of the present disclosure.

FIG. 24b is a cross-sectional view of the exemplary aspiration catheter of FIG. 23 with a tissue encroachment prevention assembly in a deployed configuration or state according to an implementation of the present disclosure.

FIG. 25a illustrates a cross-sectional view of a portion of the exemplary aspiration catheter useable with the tissue encroachment prevention assembly of FIGS. 23-24b according to an implementation of the present disclosure.

FIG. 25b illustrates a cross-sectional view of a portion of another exemplary aspiration catheter useable with the tissue encroachment prevention assembly of FIGS. 23-24b according to an implementation of the present disclosure.

FIGS. 26a-26b illustrate use of the exemplary aspiration catheter of FIGS. 23-25b within a body lumen aspirating thrombus, with the tissue encroachment prevention assembly preventing tissue damage according to an implementation of the present disclosure.

FIG. 27 is a perspective view of an exemplary aspiration catheter comprising another tissue encroachment prevention assembly according to an implementation of the present disclosure.

FIGS. 28a-28b illustrate use of the exemplary aspiration catheter of FIG. 27 within a body lumen aspirating thrombus, with the tissue encroachment prevention assembly preventing tissue damage according to an implementation of the present disclosure.

FIG. 29a illustrates a cross-sectional view of a portion of another exemplary aspiration catheter useable with another tissue encroachment prevention assembly, similar to that of tissue encroachment prevention assembly of FIGS. 23-5b, 25b in a pre-deployed configuration of state according to an implementation of the present disclosure.

FIG. 29b illustrates a cross-sectional view of a portion of another exemplary aspiration catheter useable with another tissue encroachment prevention assembly, similar to that of tissue encroachment prevention assembly of FIGS. 23-5b, 25b in a deployed configuration of state according to an implementation of the present disclosure.

FIG. 30a illustrates a cross-sectional view of a portion of the exemplary aspiration catheter usable with the tissue encroachment prevention assembly of FIGS. 29a-29b according to an implementation of the present disclosure.

FIG. 30b illustrates a cross-sectional view of a portion of another exemplary aspiration catheter usable with the tissue encroachment prevention assembly of FIGS. 29a-29b according to an implementation of the present disclosure.

FIGS. 31a-31b illustrate use of the exemplary aspiration catheter of FIG. 29a-29b within a body lumen aspirating thrombus, with the tissue encroachment prevention assembly preventing tissue damage according to an implementation of the present disclosure.

FIG. 32 is a perspective view of an exemplary aspiration catheter comprising another tissue encroachment prevention assembly according to an implementation of the present disclosure.

FIG. 33 is a perspective view of an exemplary aspiration catheter comprising another tissue encroachment prevention assembly according to an implementation of the present disclosure.

FIGS. 34a-34b illustrate use of the exemplary aspiration catheter of FIG. 33 within a body lumen aspirating thrombus, with the tissue encroachment prevention assembly preventing tissue damage according to an implementation of the present disclosure.

FIG. 35a is a perspective view of an exemplary aspiration catheter comprising another tissue encroachment prevention assembly according to an implementation of the present disclosure.

FIG. 35b is an end view of the aspiration catheter and tissue encroachment prevention assembly of FIG. 35a.

FIG. 36a is a cross-sectional view of an exemplary aspiration catheter with a tissue encroachment prevention assembly in a retracted configuration or state according to an implementation of the present disclosure.

FIG. 36b is a cross-sectional view of the exemplary aspiration catheter of FIG. 36a in a deployed configuration or state according to an implementation of the present disclosure.

FIGS. 37a-37c illustrate use of the exemplary aspiration catheter of FIGS. 36a and 36b within a body lumen aspirating thrombus, with the tissue encroachment prevention assembly preventing tissue damage according to an implementation of the present disclosure.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to preventing tissue, such as tissue of a lumen receiving an aspiration catheter, from being drawn into an aspiration lumen of the aspiration catheter and coming into close proximity to an orifice for jetted fluid from one or more supply lumens during aspiration. By preventing the tissue from being brought towards the fluid jetted or ejected from the orifice through the tissue encroachment prevention assembly, or by a means for preventing the blood vessel from being drawn into the aspiration lumen, tissue damage is prevented during aspiration of a thrombus. Thus, the aspiration process is safer for a patient.

While the present disclosure will describe a particular implementation of preventing tissue from being drawn into an aspiration lumen of the aspiration catheter and coming into close proximity to an orifice for jetted fluid from one or more supply lumens during aspiration, it should be understood that the devices, systems, and method described herein may be applicable to other uses. Additionally, elements described in relation to any embodiment depicted and/or described herein may be combinable with elements described in relation to any other embodiment depicted and/or described herein.

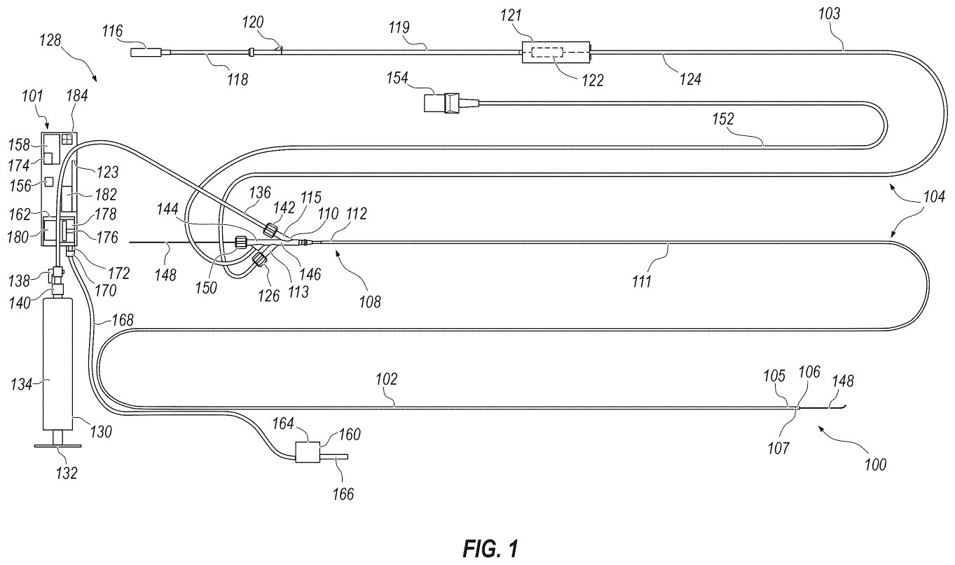

A system 100 for aspirating thrombus is illustrated in FIGS. 1-5. The system 100 for aspirating thrombus includes a pump 101, an aspiration catheter 102, and a tubing set 103. The aspiration catheter 102 and the tubing set 103 represent disposable components 104, and the pump 101, and the pump's associated pump base, is a reusable component. It is not necessary to sterilize the pump 101 as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled).

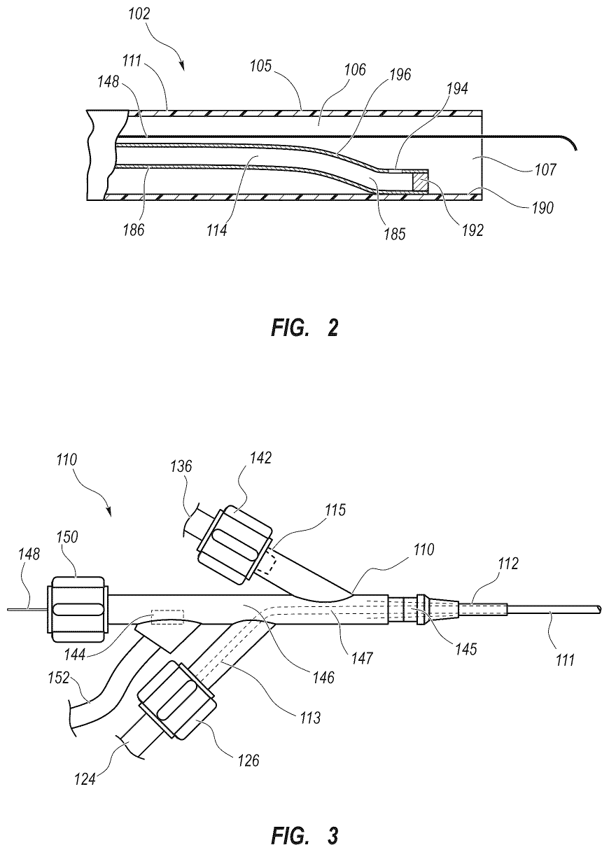

The aspiration catheter 102 has a distal end 105 and includes an over-the-wire guidewire lumen/aspiration lumen 106 extending between an open distal end 107, and a proximal end 108 comprising a y-connector 110. The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 includes a first female luer 113 which communicates with a catheter supply lumen 114 (FIG. 2), and a second female luer 115 which communicates with the guidewire lumen/aspiration lumen 106. While reference is made to the y-connector 110 communicating with the supply lumen 114 (FIG. 2), it will be understood that one or more supply lumens 114 can be included and the y-connector 110, or other connectors, can communicate with the one or more supply lumens 114.

A spike 116 for coupling to a fluid source 224 (e.g., saline bag, saline bottle) allows fluid to enter through an extension tubing 118 and flow into a supply tube 119. An optional injection port 120 allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

Accessories 128 are illustrated that are intended for applying a vacuum source, such as a syringe 130 having a plunger 132 and a barrel 134, to the aspiration lumen 106 of the aspiration catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 3, a pressure sensor or transducer 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector 154. The connector 154 is plugged into a socket 156 of the pump 101. Pressure related signals may be processed by a circuit board 158 of the pump 101. The pressure sensor or transducer 144 may be powered from the pump 101, via the cable 152. The accessories 128 may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump may include a controller 174 configured to receive one or more signals indicating on or off from the foot pedal 160. The controller 174 of the circuit board 158 may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure sensor or transducer 144 thus senses a negative pressure and sends a signal, causing the controller to start the motor 182 of the pump 101. As the effect via the electronics is substantially immediate, the motor 182 starts pumping almost immediately after the pedal 166 is depressed. As the pedal 166 of the foot pedal 160 is released, the controller 174 then causes the pinch valve 162 to close. The pressure sensor or transducer 144 thus senses that no negative pressure is present and the controller 174 causes the motor 182 of the pump 101 to shut off. Again, the effect via the electronics is substantially immediate, and thus the motor 182 stops pumping almost immediately after the pedal 166 is released. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the aspiration catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system 100 for aspirating thrombus. This can be an advantage both in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time, but may also be able to add durability to the vacuum line 136, for example, by not over-compressing. The foot pedal 160 may communicate with the pinch valve 162 via a wired connection through the pump 101 or may communicate with the pinch valve 162 wirelessly. Additionally, or alternatively, the pump may be controlled by buttons 184.

It should be noted that in certain embodiments, the pinch valve 162 and the foot pedal 160 may be incorporated for on/off operation of the pinch valve 162 on the vacuum line 136, without utilizing the pressure sensor 144. In fact, in some embodiments, the pressure sensor 144 may even be absent from the system 100 for aspirating thrombus, the foot pedal 160 being used as a predominant control means.

Turning to FIG. 2, a supply tube 186, which contains the catheter supply lumen 114, freely and coaxially extends within the over-the-wire guidewire lumen/aspiration lumen 106. At least a distal end 185 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 185 of the supply tube 186. The plug 192 blocks the exit of pressurized fluid, and thus the pressurized fluid is forced to exit through an orifice 194 in the wall 196 of the supply tube 186. The free, coaxial relationship between the supply tube 186 and the catheter shaft 111 along their respective lengths, allows for improved flexibility. In some embodiments, in which a stiffer proximal end of the aspiration catheter 102 is desired (e.g., for pushability or even torqueability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

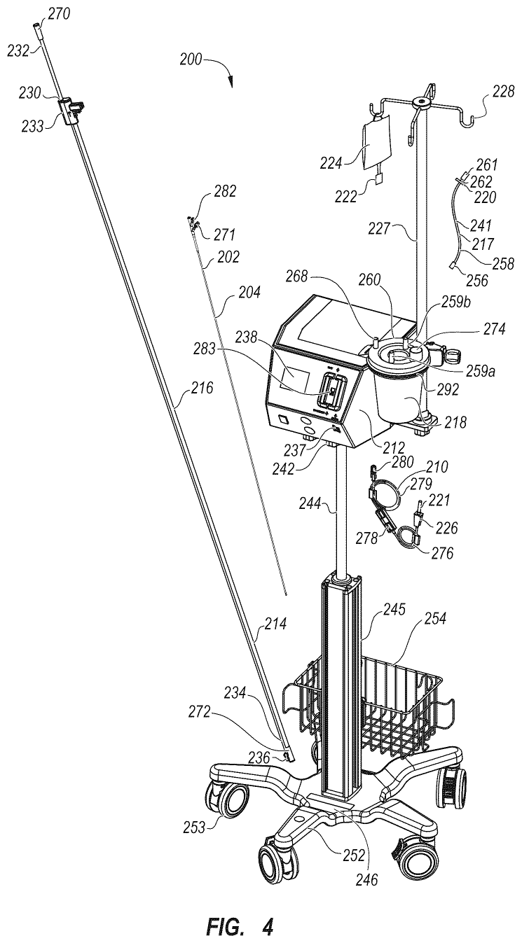

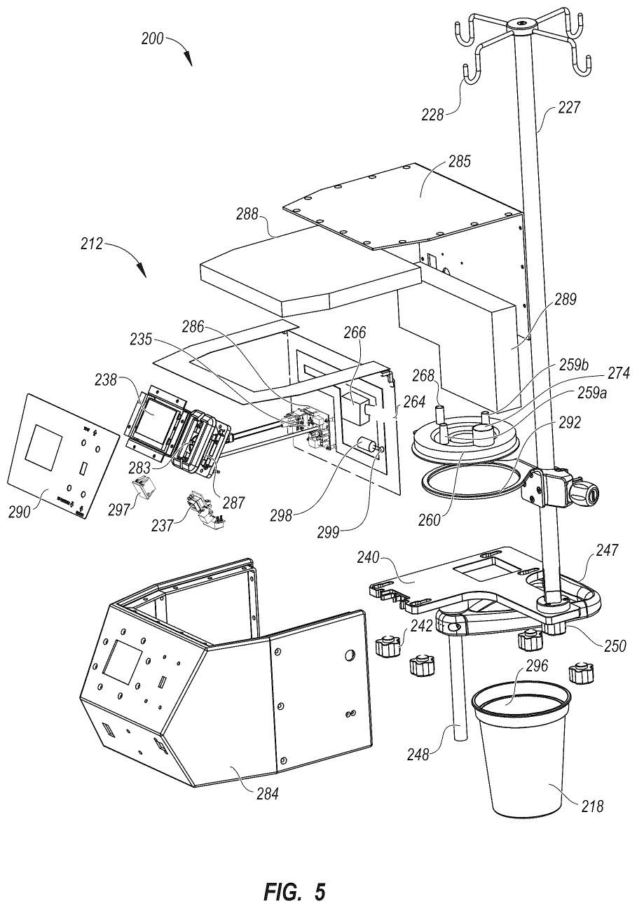

A system 200 for aspirating thrombus is illustrated in FIGS. 4-5. The system 200 for aspirating thrombus is similar to the system 100 and so the disclosure related to the system 100 is also applicable to the description of system 200. An aspiration catheter 202 is similar to the aspiration catheter 102 of FIGS. 1-3 and as such the description related to the aspiration catheter 102 of FIGS. 1-3 is also applicable to the description of the aspiration catheter 202.

The aspiration catheter 202 is configured for aspirating thrombus from peripheral vessels, but may also be configured with a size for treating coronary, cerebral, pulmonary or other arteries, or veins. The aspiration catheter 202/system 200 may be used in interventional procedures, but may also be used in surgical procedures. The aspiration catheter 202/system 200 may be used in vascular procedures, or non-vascular procedures (other body lumens, ducts, or cavities). The catheter 202 comprises an elongate shaft 204 configured for placement within a blood vessel of a subject; a catheter supply lumen 114 (FIG. 3) and a guidewire/aspiration lumen 106, each extending along the shaft, the supply lumen 114 having a proximal end 147 and a distal end 185, and the aspiration lumen 106 having a proximal end 145 (FIG. 3) and an open distal end 107 (FIG. 2); and an orifice or opening 194 at or near the distal end 185 of the supply lumen 114, the opening configured to allow the injection of pressurized fluid into the aspiration lumen 106 at or near the distal end 107 of the aspiration lumen 106 when the pressurized fluid is pumped through the supply lumen 114. In some embodiments, the orifice or opening 194 may be located proximal to the distal end 185 of the supply lumen 114. In some embodiments, the distal end 185 of the supply lumen 114 may comprise a plug 192. A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. The pressure sensor 230 is shown in FIG. 4 within a lumen at a junction between a first aspiration tube 232 and a handpiece 233. A cable 234 carries signals output from the pressure sensor 230 to a controller 235 (FIG. 5) in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In alternative embodiments, the pressure sensor 230 may be replaced by another type of sensor that is configured to characterize fluid flow. In some embodiments, the sensor is a flow sensor, such as a Doppler flow velocity sensor.

The SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

In use, a user connects a first connector 256 at a first end 258 of the non-sterile suction tubing 217 to a second port 259b on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218. Alternatively, the vacuum inside the canister 218 may be maintained manually, without a vacuum pump, by evacuating the canister 218 via one or more additional ports 268. A user connects a first connector 270 of the sterile suction tubing 216 to an aspiration luer 271 of the aspiration catheter 202 (similar to luer 115), and connects the second connector 272 of the sterile suction tubing 216 to port 259a in the lid 260 of the canister 218. Connector 236 is then coupled to the mating receptacle 237 in the SDU 212 for communication with the handpiece 233 and/or the pressure sensor 230. For instance, the connector 236 can be snapped into mating receptacle 237 in the SDU 212 for communication with elements of the handpiece 233 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 278 is snapped in place, keeping the internal contents (e.g., saline) sterile. Systems configured for performing this type of sterile injection of high-pressure saline are described in U.S. Pat. No. 9,883,877, issued Feb. 6, 2018, and entitled, “Systems and Methods for Removal of Blood and Thrombotic Material”, which is incorporated by reference in its entirety for all purposes. The SDU 212 is enclosed within a case 284 and a case lid 285. The controller 235 (FIG. 5) may reside on a circuit board 286. Noise from a motor 287 controlling the saddle 283 and from the vacuum pump 266 is abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 278 may couple to the saddle 283 by clipping, friction fitting, vacuum fitting, or other means.

FIG. 5 illustrates aspects pertaining to the vacuum canister 218, in which aspirant (e.g., thrombus, blood, saline) that is evacuated from the patient through the aspiration lumen 106 is collected. The canister 218 may be held in a canister mount 292 carried by the IV pole 227, or alternatively carried by any other part of the system 200. A lid 260 is configured to cover a portion of the canister 218, such as in a snapping manner, to close an interior 296 of the canister 218. Alternatively, the lid 260 may couple to the canister 218 by screwing, clipping, friction fitting, or other means.

The lid 260 may comprise two or more ports, including the first port 259a and second port 259b for providing negative pressure/vacuum to the aspiration lumen 106. For example, the lid 260 may comprise two ports, three ports, four ports, or more than four ports. Sterile suction tubing 216 may be connected to the lid 260 of the vacuum canister 218 at a first port 259a for transmitting a negative pressure to the sterile suction tubing 216 and to the aspiration lumen 106 of the aspiration catheter 102. Non-sterile suction tubing 217 may be connected to the lid 260 of the vacuum canister 218 at a second port 259b for providing a negative pressure to the vacuum canister 218. A negative pressure may be provided to the non-sterile suction tubing 217 (and to sterile suction tubing 216 and the aspiration lumen 106 connected therewith) by a vacuum source (e.g., a vacuum pump or syringe). The system 200 may also comprise means for sealing the two or more ports of the lid 260 when not in use, such as one or more port caps. A filter may be placed over an entry to the second port 259b so as to prevent aspirant from traveling along the non-sterile suction tubing 217 from the vacuum canister 218 to the vacuum source.

The vacuum canister 218 preferably has a sufficient volumetric capacity for receiving all aspirant collected during the surgical procedure. Receptacles having a volumetric capacity of approximately 100 cubic inches (about 1638.71 cubic centimeters), or receptacles having a diameter of approximately 5.0 inches (about 12.70 centimeters) and a height of approximately 7.0 inches (about 17.78 centimeters), have been found to provide sufficient volumetric capacity.

Returning to FIG. 5, a solenoid 298 is carried internally in the SDU 212, and is configured to interface with the interior 296 of the canister 218, via the suction tubing 214, or via any additional tubing. The solenoid 298 is configured to vent the negative pressure inside the canister 218, by opening a valve 299 coupled to the solenoid (mechanically or electromagnetically) that opens the interior 296 of the canister 218 to ambient pressure. The venting allows any foaming of blood or fluid, such as any aspirated liquid, within the canister 218 to be reduced. Foaming can occur during a thrombolysis procedure due to cavitation, as air bubbles are formed. The solenoid 298 is then configured to close the valve 299, to allow negative pressure to again be built up within the interior 296 of the canister 218. The controller 235 is configured to automatically energize the solenoid 298, in order to allow for the degassing/defoaming. For example, the controller 235 may send a signal to energize the solenoid 298 based on the measurement of a targeted negative pressure and/or a targeted time of aspiration cycle. In other cases, the controller 235 can send a signal to energize the solenoid 298 every minute, every five minutes, every ten minutes, etc. Additionally, a user can operate the controller 235, and more generally the controller 174, of the system 200 through the interface panel 290 to initiate degassing/defoaming of the interior 296. The venting may also be able to remove air bubbles inside the other lumens of the catheter and tubing sets.

In some embodiments, the controller 235 can output or send a signal to energize the solenoid 298 to open the valve 299, in order to stop any aspiration, while still allowing the SDU 212 to deliver saline, medication, or saline combined with medication (e.g., thrombolytic drugs), so that the fluids can be delivered out of the open distal end 107 (instead of being aspirated through the aspiration lumen 106).

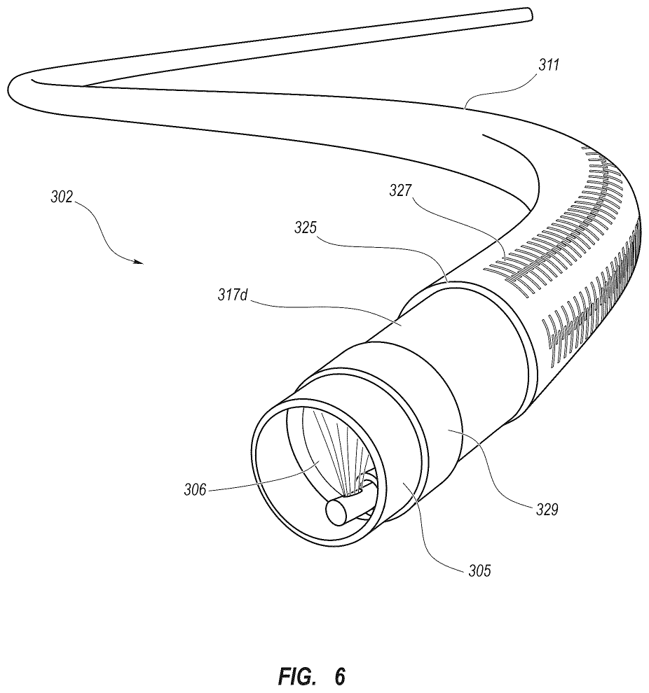

Turning to FIGS. 6-8 illustrated is another configuration of an aspiration catheter 302 that can be used with the systems 100 or 200 for aspirating thrombus. As such, the discussions related to the aspiration catheter 102 and the aspiration catheter 202 are also applicable to the aspiration catheter 302 illustrated in FIGS. 6-8.

As shown in more detail in FIGS. 6-8, aspiration catheter 302 includes an aspiration lumen 306 formed by a shaft 311, such as a hypotube, jacketed by a polymer jacket or a polymer jacket is laminated on the hypotube. For instance, a shaft body 317a is illustrated being jacket by a jacket 317b. A distal end 305 of the aspiration catheter 302 includes a multilayer structure. A portion 317c of the jacket 317b extends distally of a shaft distal end 325 of the shaft 311 to form part of the distal end 305. An outer layer or outer jacket 317d overlaps the jacket 317b, extends towards and overlaps a shaft distal end 325, and forms the aspiration catheter distal end 305a or the distal most end of the aspiration catheter with the distal opening 307. The outer jacket 317d protects a distal portion 305 of a supply tube 386 containing a supply lumen 314. While reference is made of a multilayer structure, it will be understood that one or more layers can be omitted or combined together. Additionally, one or more of the layers or shaft body can include braided or other members to increase strength and/or flexibility. Alternatively, the shaft and associated layers can be formed by extruding the shaft or using other structures to form the shaft. Alternatively, in other configurations one or more supply lumens 314, and one or more supply tubes 386, can be associated with the shaft 311.

The supply lumen 314 may be configured to provide a high pressure fluid injection, such as saline, within the aspiration lumen 306 for macerating a thrombus as it is aspirated, such as illustrated in FIG. 8 where the aspiration catheter 302 is disposed within a vessel lumen VL of a vessel V and aspirant A is being drawing into the aspiration lumen 306. The saline injection may occur through orifice 394 near the distal end of the supply lumen 314 if the opening of the supply lumen is plugged with a plug similar to plug 192 (FIG. 2). Aspiration catheter 302 may also include a radiopaque (RO) ring 329 at or near the distal end 305 of aspiration catheter 302 for identifying the location of aspiration. The radiopaque ring 329 optionally encircles the aspiration catheter 302. In the illustrated configuration, the RO ring 329 is disposed between the jacket 317b and outer jacket 317d. The RO ring 329 can be formed of any suitable radiopaque material, such as tantalum, tungsten, platinum/iridium, gold, silver, and combinations or modifications thereof.

The radiopaque ring 329 also aids with reducing the likelihood of the distal end 305 collapsing during movement through the circulatory vessels where the distal end 305 can encounter vascular tortuosity. With the outer jacket 317d extending to the distal opening 307, flexibility of the distal end 305 is maintained. The radiopaque ring 329 provides structure to keep the distal opening 307 open and prevent the outer jacket 317d and jacket 317b collapsing during jetting and/or aspiration.

The shaft 311 can include one or more openings 327 to increase a flexibility of shaft 311 to aid with advancement of the aspiration catheter 302 through the tortuous anatomy of a patient. While reference is made to a “hypotube,” it will be understood that other tubular structures can be used for the shaft 311. Additionally, the shaft 311 can be formed from polymers, metals, alloys, braided structures, coiled structures, and combinations or modifications thereof. Furthermore, the jacket 317b and outer jacket 317d can be formed of a variety of polymers and copolymers, plastics, PEBAX, HYTREL, rubber, nylon, polyethylene, polyurethane, polyester and combinations or modifications thereof.

In some situations, during aspiration, damage to the vessel might occur because the vessel wall is drawn into the aspiration lumen 306 and comes into contact with the pressurized fluid injection (e.g., a high pressure saline spray at, for example, 650 psi) from supply lumen 314. Therefore, a tissue encroachment prevention assembly can prevent aspiration catheter 302 from damaging the vessel during aspiration. The tissue encroachment prevention assembly can be selectively added to the distal end 305 of the aspiration catheter 302 or the distal end 305 can be modified or changed to accommodate the tissue encroachment prevention assembly.

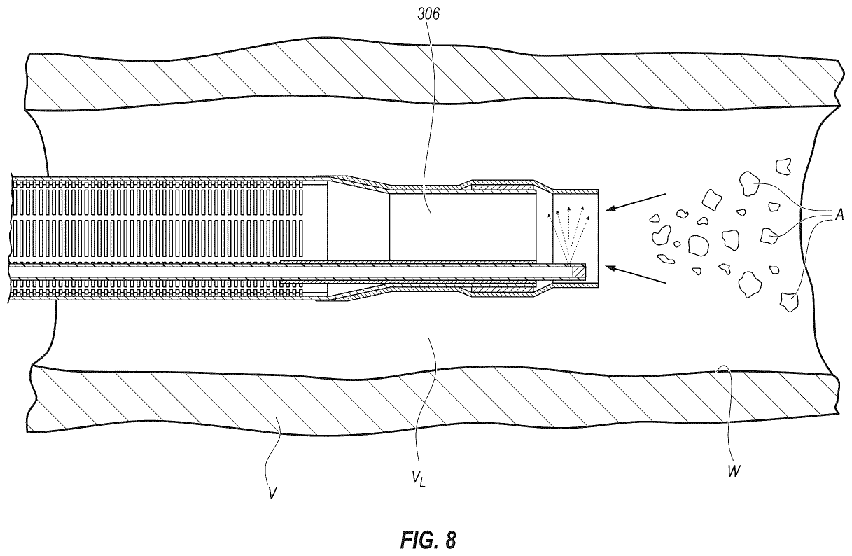

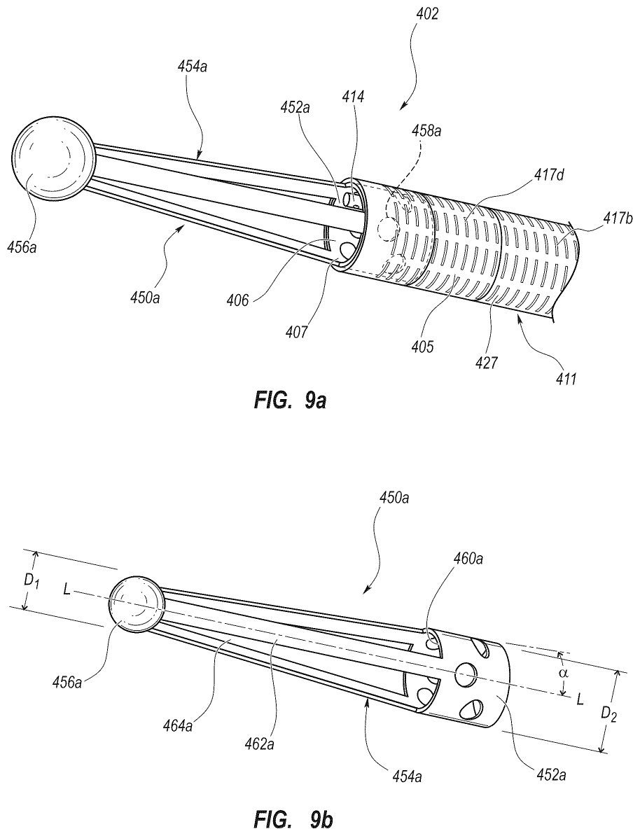

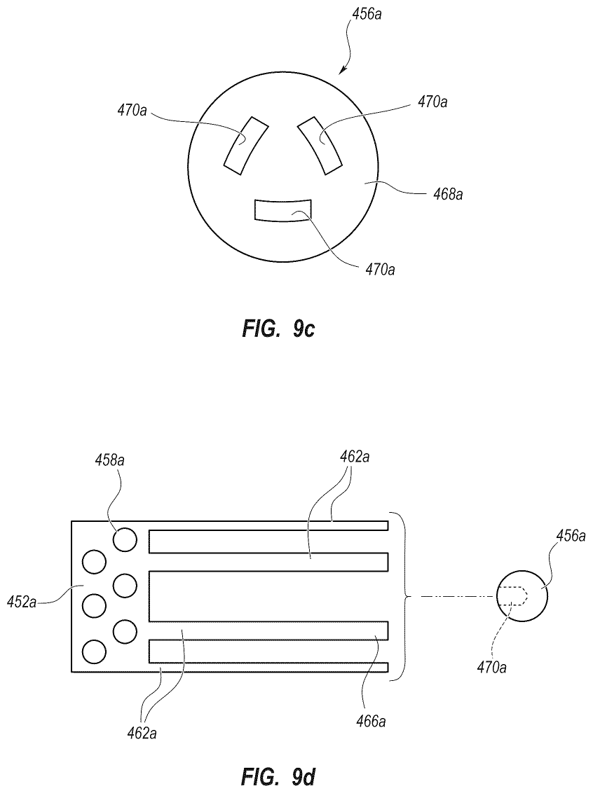

FIGS. 9a-9d generally illustrate tissue encroachment prevention assemblies that provide a barrier to tissue being drawn into an aspiration lumen. In this way, access to the aspiration lumen of the aspiration catheter is controlled or limited so that tissue does not encroach or is not drawn into the aspiration lumen and towards the high pressure spray from the supply lumen, while thrombus or aspirant is still capable of being collected. Such tissue encroachment prevention assemblies may provide a means for preventing the blood vessel from being drawn into the aspiration lumen and coming into close proximity to an orifice of a supply lumen during aspiration.

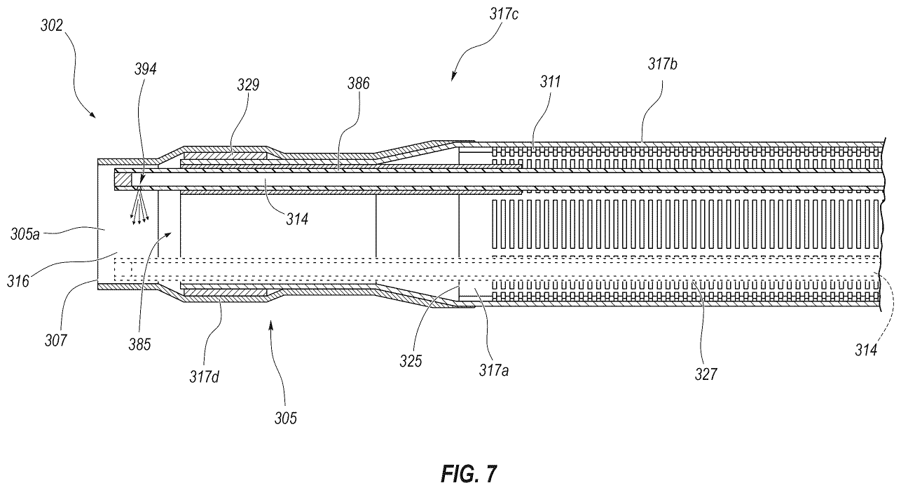

As illustrated in FIG. 9a, an aspiration catheter 402 includes a shaft 411 with a tissue encroachment prevention assembly 450a disposed towards a distal end 405 of the shaft 411, a portion of the tissue encroachment prevention assembly 450a optionally extending from and/or forming a distal opening 407 of the aspiration catheter 402. The shaft 411 includes one or more openings 427 to aid with flexibility of the shaft 411 and the distal end 405. A jacket 417b extends along the shaft 411, with an outer jacket 417d being disposed over a portion of the jacket 417b. Unlike aspiration catheter 302 (FIG. 7), a portion of a tissue encroachment prevention assembly 450a is disposed between the jacket 417b and the outer jacket 417d and functions as a radiopaque (RO) ring. While reference is made to the tissue encroachment prevention assembly 450a being disposed between the jacket 417b and the outer jacket 417d, it will be understood that the tissue encroachment prevention assembly 450a can be disposed at various other positions on the aspiration catheter 402. For instance, the tissue encroachment prevention assembly 450a can be disposed between the shaft 411 and one of the jacket 417b and the outer jacket 417d, such as when one of the jacket 417b and the outer jacket 417d is included. For instance, the outer jacket 417d can be omitted and the jacket 417b can extend to a distal end of the shaft 411 with the tissue encroachment prevention assembly 450a attached to the shaft. In still another configuration, the tissue encroachment prevention assembly 450a can be mounted or otherwise disposed on the outside of the outer jacket 417d. In still another configuration, such as when the aspiration catheter 402 does not include one or both of the jacket 417b and the outer jacket 417d, the tissue encroachment prevention assembly 450a can be disposed on an outermost surface of the aspiration catheter 402 and/or the shaft 411.

As illustrated in 9a and 9b, the tissue encroachment prevention assembly 450a comprises a base 452a, having a generally cylindrical form or ring-like form, from which extends a tine assembly 454a terminating in an atraumatic member 456a (FIG. 9d illustrates the tissue encroachment prevention assembly 450a in a flat profile with the atraumatic member 456a detached). The combination of the base 452a, the tine assembly 454a, and the atraumatic member 456a forms a cage-like structure that allows clot, thrombus, aspirant, blood, etc. to flow into the aspiration lumen 406 while preventing vessel tissue from being drawing into the aspiration lumen 406. The base 452a can optionally form, at least in part, the distal opening 407 of the aspiration catheter 402. The combination of the tine assembly 454a and the atraumatic member 456a prevents the vessel wall from being pulled into the aspiration lumen 406. For instance, the tine assembly 454a may include three tines 462a, although more or less tines are also possible, in order to further prevent vessel tissue from being drawn into aspiration lumen 406 or toward, or into close proximity to, high pressure saline spray from the supply lumen 414 and associated orifice 494. In other configurations one or more supply lumens 414, and one or more supply tubes 486, can be associated with the shaft 411.

The base 452a is has a generally cylindrical form or ring-like form having holes 458a dispersed throughout the base 452a that aid with mounting the base 452a between the jacket 417b and the jacket 417d, and more generally mounting the base to the distal end 405 of the shaft 411. In this illustrated configuration, the polymer forming the jacket 417d flows into the holes 458a to join the base 452a to the shaft 411. Optionally, the polymer forming the jacket 417b can also flow or reflow into the holes 458a of the base 452a. In either case, the base 452a becomes joined, coupled, or mounted to the shaft 411. It will be understood that instead of flowing or reflowing the material forming the jackets 417b, 417d, the base 452a can be mounted to the shaft 411a using adhesives, glues, thermal bonding, laser welding, welding, soldering, mechanical fasteners, such as rivets, detents, threads, modifications or combinations thereof, crimping, or combinations and modifications thereof.

The base 452a has an internal diameter ranging from about 2 mm to about 3 mm, from about 3 mm to about 4 mm, from about 4 mm to about 6 mm, from about 6 mm to about 8 mm, from about 2 mm to about 8 mm, or is in a range between any two of the foregoing. The base 452a has an outside diameter ranging from about 2.2 mm to about 3.2 mm, from about 3.2 mm to about 4.2 mm, from about 4.2 mm to about 6.2 mm, from about 6.2 mm to about 8.2 mm, from about 2 mm to about 9 mm, or is in a range between any two of the foregoing. The base 452a has a length ranging from about 1 mm to about 2 mm, from about 2 mm to about 4 mm, from about 4 mm to about 6 mm, from about 6 mm to about 8 mm, about 1 mm to about 8 mm, or is in a range between any two of the foregoing. Stated another way, the base 452a has an internal diameter ranging from about 0.079″ to about 0.118″, from about 0.118″ mm to about 0.157″, from about 0.157″ to about 0.236″, from about 0.236″ to about 0.315″, from about 0.079″ to about 0.315″, or is in a range between any two of the foregoing. The base 452a has an outside diameter ranging from about 0.087″ to about 0.126″, from about 0.126″ to about 0.165″, from about 0.165″ to about 0.244″, from about 0.244″ to about 0.323″, from about 0.118″ to about 0.354″, or is in a range between any two of the foregoing. The base 452a has a length ranging from about 0.039″ to about 0.079″, from about 0.079″ to about 0.157″, from about 0.157″ to about 0.236″, from about 0.236″to about 0.315″, about 0.039″ to about 0.315″, or is in a range between any two of the foregoing.

Extending from a distal end 460a of the base 452a is the tine assembly 454a. The tine assembly 454a includes tines 462a having a generally elongate form and a generally rectangular cross-section (although circular, oval, square, polygonal, and combinations or modifications thereof are also possible), as shown in FIG. 9c. The tines are inclined toward the atraumatic member 456a, with the atraumatic member 456a having a dimension D1 across or transverse to a longitudinal axis L of the tissue encroachment prevention assembly 450a that is smaller than a dimension D2 across or transverse to the longitudinal axis L of the base 452a. This configuration aids with steering the aspiration catheter 402 and provides gaps 464a that allow aspirant and thrombus to pass, but prevent tissue from being drawn into the aspiration lumen 406. Since a width of the tines 462a taper towards the atraumatic member 456a, the gaps 464a also form a tapered configuration. However, this need not be the case and the gaps 464a can have various shapes and forms to prevent passage of tissue.

An angle of inclination of the tines 462a in relation to the longitudinal axis L can vary based upon at least a length of the tines 462a. Longer tines provide increased flexibility to the tine assembly 454a, while also providing larger gaps 464a. Similarly, shorter tines provide decreased flexibility to the tine assembly 454a, while providing smaller gaps 464a. As such, depending upon the particular elasticity of the vessel tissue, different configurations of the tissue encroachment prevention assembly 450a are possible. For instance, a length of the tines can range from about 5 mm to about 6 mm, from about 6 mm to about 10 mm, from about 10 mm to about 20 mm, from about 20 mm to about 25 mm, from about 5 mm to about 25 mm, or is in a range between any two of the foregoing. Stated another way, a length of the tines can range from about 0.2″ to about 0.24″, from about 0.24″ to about 0.39″, from about 0.39″ to about 0.79″, from about 0.79″ to about 0.98″, from about 0.2″ to about 0.98″ mm, or is in a range between any two of the foregoing. An angle α between a tine and the longitudinal axis L can range from about 5 degrees to about 30 degrees, from about 30 degrees to about 45 degrees, from about 45 degrees to about 60 degrees, from about 5 degrees to about 60 degrees, or is in a range between any two of the foregoing.

Attached to distal ends 466a of the tines 462a is the atraumatic member 456a. In this particular configuration, the atraumatic member 456a includes a generally spherical ball 468a having apertures 470a to receive the distal ends 466a, such as illustrated in FIGS. 9c and 9d. The apertures 470a can be blind holes to receive the distal ends 466a. For instance, when the tissue encroachment prevention assembly 450a is laser cut from a hypotube, each distal end 466a of each tine 462a is disposed in a respective aperture 470a and fixed in place, such as through welding, laser welding, soldering, mechanical fastening, crimping, thermal bonding, adhesive bonding, brazing, or combination or modifications thereof. While reference is made to individual apertures 470a receiving individual distal ends 466a of the tines 462a, it will be understood that two or more of the distal ends 466a of the tines 462a can be received in one aperture 470a. Additionally, one skilled in the art would understand that the point of attachment for the tines 462a need not be a spherical ball—it need only be an element or configuration which helps to form a cage structure with the tines and which is atraumatic. For instance, the distal ends 466a can be joined together to form a “ball-like” form or other structure providing an atraumatic end to the tissue encroachment prevention assembly 450a.

As mentioned above, the base 452a (or more generally the tissue encroachment prevention assembly 450a) can replace the radiopaque ring 329 (FIG. 7-8). For example, the base 452a and tines 462a can be formed of stainless steel, with the atraumatic member 456a being made of a radiopaque material, such as a material or alloy containing a material with a higher atomic mass such as tantalum, tungsten, platinum/iridium, gold, silver, and so forth. Alternatively, or in addition to, the atraumatic member 456a being made of a radiopaque material, one or more of the base 452a and/or the tines 462a can be formed of a radiopaque material. In still another configuration, a radiopaque coating can be applied to one or more parts of the tissue encroachment prevention assembly 450a, such as one or more of the base 452a, the tines 462a, and/or the atraumatic member 456a. In other configurations, the base 452a, the tines 462a, and/or the atraumatic member 456a is formed of a metal, alloy, shape-memory material, composite, polymer, or combinations or modifications thereof, whether or not the same is radiopaque.

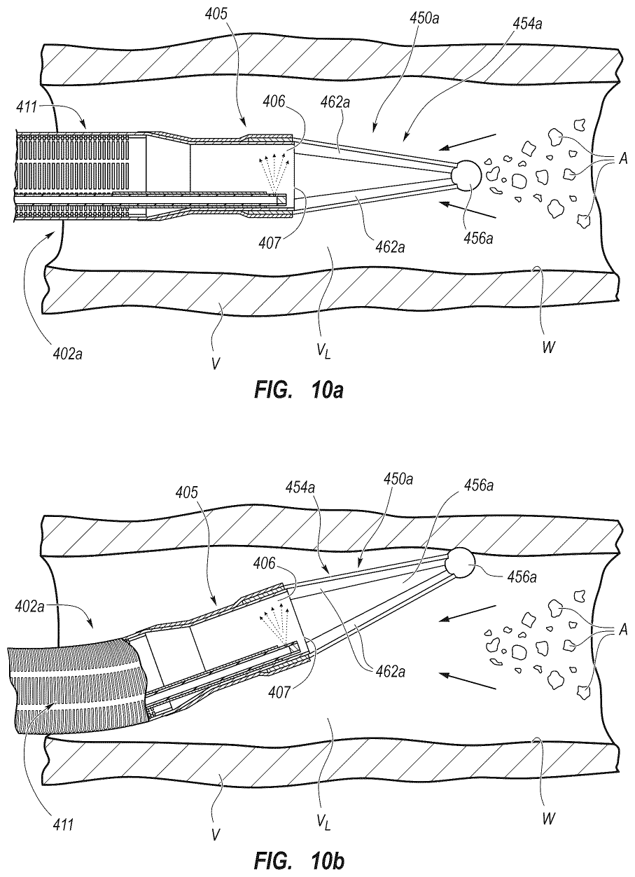

Turning to FIGS. 10a and 10b, the tissue encroachment prevention assembly 450a is illustrated preventing tissue being drawn into the aspiration lumen 406. As the aspiration catheter 402 is moved within the vessel V during aspiration of the clot or other aspirant A, the distal end 405 can move across the vessel lumen VL. Without the tissue encroachment prevention assembly 450a, the distal end 405 of the shaft 411 could contact the wall W and the vessel tissue can be drawn into the aspiration lumen 406. However, with the inclusion of the tissue encroachment prevention assembly 450a, the atraumatic member 456a contacts the wall W and maintains a separation of the aspiration lumen 406 and the wall tissue. This prevents tissue damage through vessel tissue being drawn into close proximity to high pressure saline spray from the supply lumen or one or more supply lumens. However, inclusion of the tine assembly 454a allows passage of clot or aspirant A through the gaps 464a and into the aspiration lumen 406 thereby allowing aspiration to continue. Additionally, a resiliency, flexibility, spring force, and elasticity of the tissue encroachment prevention assembly 450a can provide a rebound force to move the distal end 405, and thereby the distal opening 407, away from the aspiration lumen 406 so as to prevent damage to the wall or vessel tissue. Reference has been made to various structures and configurations of tissue encroachment prevention assemblies in relation to tissue encroachment prevention assembly 450a. However, it will be understood that various other possible structures and configurations are also possible.

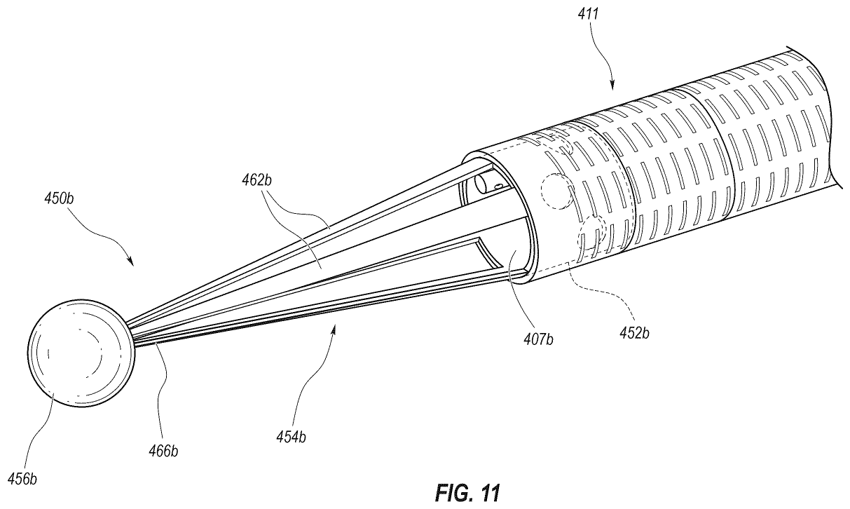

Turning to FIG. 11, illustrated is another tissue encroachment prevention assembly 450b that provides a barrier to tissue being drawn into an aspiration lumen and, thus, access to the aspiration lumen of the aspiration catheter is controlled or limited such that tissue does not encroach or be drawn into the aspiration lumen and towards the high pressure spray from the supply lumen or one or more supply lumens, while thrombus or aspirant is still capable of being collected. Such tissue encroachment prevention assembly may provide a means for preventing the blood vessel from being drawn into the aspiration lumen and coming into close proximity to an orifice of a supply lumen (or orifices of one or more supply lumens) during aspiration. The tissue encroachment prevention assembly 450b is similar to the tissue encroachment prevention assembly 450a and so the disclosure related to the tissue encroachment prevention assembly 450a is also applicable to the description of tissue encroachment prevention assembly 450b. Like reference numerals may be utilized to designate corresponding or similar parts.

As illustrated, the tissue encroachment prevention assembly 450b extends from the shaft 411 and includes a base 452b having a generally cylindrical form or ring-like form, a tine assembly 454b extending from the base 452b, and an atraumatic member 456b disposed or arranged at a distal end of the tissue encroachment prevention assembly 450b. The combination of the base 452b, the tine assembly 454b, and the atraumatic member 456b forms a cage-like structure that allows clot, thrombus, aspirant, blood, etc. to flow into the aspiration lumen 406 and prevent vessel tissue from being drawn into the aspiration lumen 406. A portion of the tissue encroachment prevention assembly 450b optionally extends from and/or forms a distal opening 407b of the aspiration catheter 402. The tines 462b forming the tine assembly 454b taper from the base 452b towards the atraumatic member 456b that is mounted to distal ends 466b of the tines 462b. This configuration provides more open space towards the atraumatic member 456b in order to allow a clot to be easily drawn in, while preventing the vessel tissue from being drawn into close proximity to the high pressure saline spray from the supply lumen.

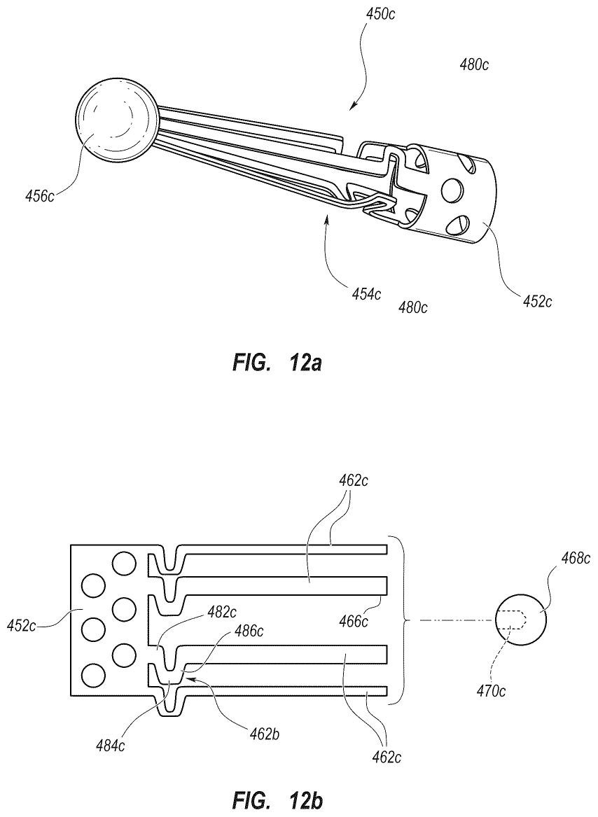

Turning to FIGS. 12a-12b, illustrated is another tissue encroachment prevention assembly 450c that provides a barrier to tissue being drawn into an aspiration lumen and so access to the aspiration lumen of the aspiration catheter is controlled or limited such that tissue does not encroach or be drawn into the aspiration lumen and towards the high pressure spray from the supply lumen or one or more supply lumens, while thrombus or aspirant is still capable of being collected. Such tissue encroachment prevention assembly may provide a means for preventing the blood vessel from being drawn into the aspiration lumen and coming into close proximity to an orifice of a supply lumen during aspiration. The tissue encroachment prevention assembly 450c is similar to the tissue encroachment prevention assemblies 450a and 450b and so the disclosure related to the tissue encroachment prevention assemblies 450a and 450b is also applicable to the description of tissue encroachment prevention assembly 450c. Like reference numerals may be utilized to designate corresponding or similar parts.

As illustrated, the tissue encroachment prevention assembly 450c includes a base 452c, a tine assembly 454c extending from the base 452c having a generally cylindrical form or ring-like form, and an atraumatic member 456c disposed or arranged at a distal end of the tissue encroachment prevention assembly 450c. The combination of the base 452c, the tine assembly 454c, and the atraumatic member 456c forms a cage-like structure that allows clot, thrombus, aspirant, blood, etc. to flow into the aspiration lumen 406 and prevent vessel tissue from being drawing into the aspiration lumen 406. The width of the tines 462c forming the tine assembly 454c taper from the base 452c towards the atraumatic member 456c that is mounted to distal ends 466c of the tines 462c. This configuration provides more open space towards the atraumatic member 456c in order to allow a clot to be easily drawn in, while preventing the vessel tissue from being drawn into close proximity to high pressure saline spray from the supply lumen.

As illustrated in FIGS. 12a-12b, tines 462c of a tine assembly 454c include a spring member 480c. The spring member 480c allows the tines 462c to flex, making the catheter “tip”—i.e., the tissue encroachment prevention assembly 450c—“softer.” As illustrated, the spring member 480c has a generally “U” configuration with a first spring leg 482c connected to a second spring leg 486c via a curved member 484c. The second spring leg 486c connects to a remainder of the tine 462c, while the first spring leg 482c connects to the base 452c. It will be understood, however, that the spring member 480c can be intermediate a distal end 466c of the tine 462c and the base 452c and so the first spring leg 482c can also connect to the tine 462c rather than the base 452c. One skilled in the art will understand that spring member 480c can be achieved by various designs, such as a “w” configuration, an “s” configuration, or any other configuration which will allow the tines 462c to bend. Furthermore, while one spring member 480c is associated with or formed on each tine 462c (or otherwise forms part of each tine 462c) in FIGS. 12a and 12b, one or more spring members 480c can be included in each tine 462c and each tine 462c can include the same or a different number of spring members 480c from other tines 462c.

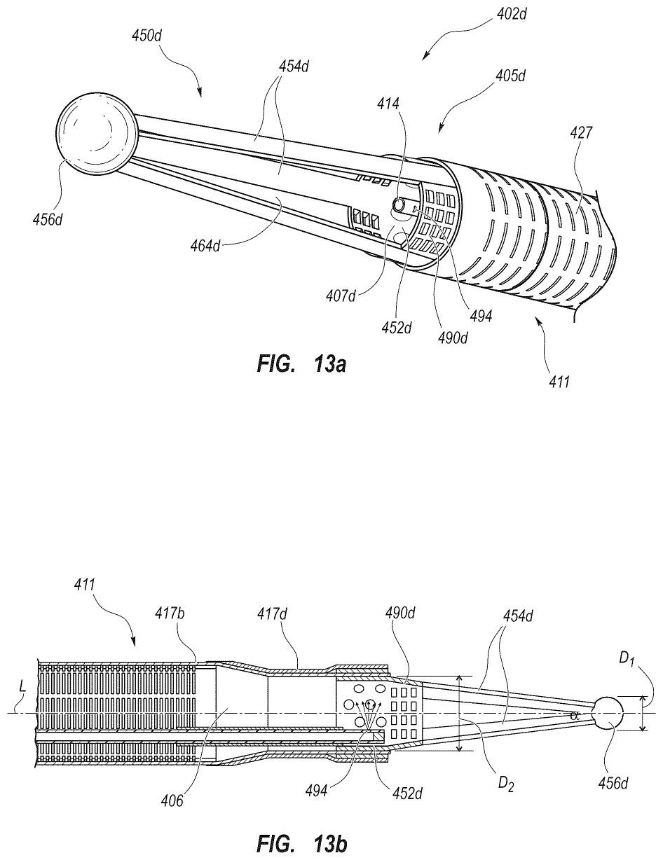

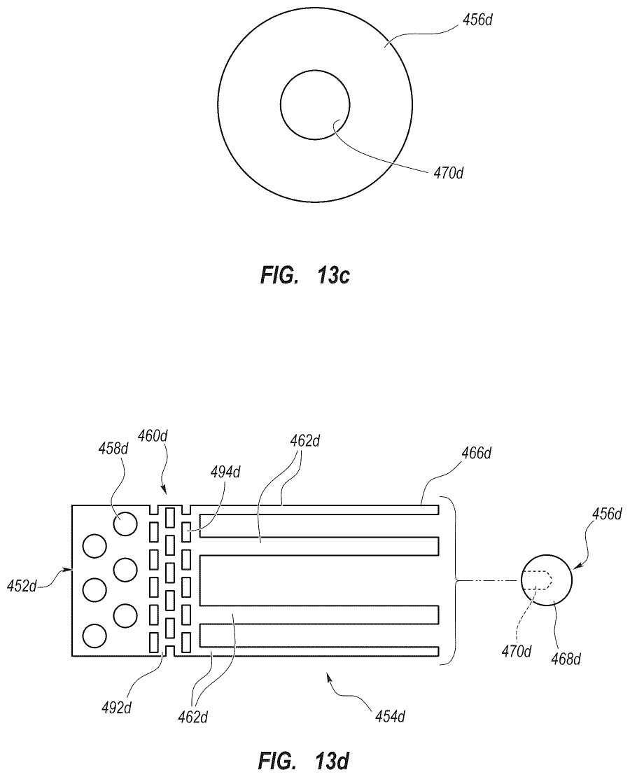

Turning to FIGS. 13a-13d, illustrated is another tissue encroachment prevention assembly 450d that provides a barrier to tissue being drawn into an aspiration lumen and thereby controls or limits access to the aspiration lumen of the aspiration catheter such that tissue does not encroach or be drawn into the aspiration lumen, and towards the high pressure spray from the supply lumen or one or more supply lumens, while thrombus or aspirant is still capable of being collected. Such tissue encroachment prevention assembly may provide a means for preventing the blood vessel from being drawn into the aspiration lumen and coming into close proximity to an orifice of a supply lumen (or orifices of one or more supply lumens) during aspiration. The tissue encroachment prevention assembly 450d is similar to the tissue encroachment prevention assemblies 450a, 450b, and 450c. Therefore, the disclosure related to the tissue encroachment prevention assemblies 450a, 450b, and 450c is also applicable to the description of tissue encroachment prevention assembly 450d. Like reference numerals may be utilized to designate corresponding or similar parts.

As illustrated in FIGS. 13a-13d, an aspiration catheter 402d includes a shaft 411 with a tissue encroachment prevention assembly 450d disposed towards a distal end 405d of the shaft 411, a portion of the tissue encroachment prevention assembly 450d optionally extending from and/or forming a distal opening 407d of the aspiration catheter 402d. The shaft 411 includes one or more openings 427 to aid with flexibility of the shaft 411 and the distal end 405. A jacket 417b extends along the shaft 411, with an outer jacket 417d being disposed over a portion of the jacket 417b. Unlike aspiration catheter 302 (FIG. 7), a portion of the tissue encroachment prevention assembly 450d is disposed between the jacket 417b and the outer jacket 417d and functions as a radiopaque (RO) ring. While reference is made to the tissue encroachment prevention assembly 450d being disposed between the jacket 417b and the outer jacket 417d, it will be understood that the tissue encroachment prevention assembly 450a can be disposed between the shaft 411 and one of the jacket 417b and the outer jacket 417d, such as when one of the jacket 417b and the outer jacket 417d is included. For instance, the outer jacket 417d can be omitted and the jacket 417b can extend to a distal end of the shaft 411 with the tissue encroachment prevention assembly 450d attached to the shaft.

As illustrated in FIGS. 13a-13d, the tissue encroachment prevention assembly 450d comprises a base 452d, having a generally cylindrical form or ring-like form, from which extends a tine assembly 454d terminating in an atraumatic member 456d. The combination of the base 452d, the tine assembly 454d, and the atraumatic member 456d forms a cage-like structure that allows clot, thrombus, aspirant, blood, etc. to flow into the aspiration lumen 406 and prevent vessel tissue from being drawing into the aspiration lumen 406. For instance, the tine assembly 454d as shown in FIG. 13d includes three tines 462d, although more or less tines are also possible, in order to further prevent vessel tissue from being drawn into aspiration lumen 406 or toward or into close proximity to a high pressure saline spray from the supply lumen 414 and associated orifice 494.

The base 452d has a generally cylindrical form or ring-like form having holes 458dthat aid with mounting the base 452d between the jacket 417b and the jacket 417d, and more generally mounting the base to the distal end 405 of the shaft 411. In this illustrated configuration, the polymer forming the jacket 417d flows into the holes 458d to join the base 452d to the shaft 411. Optionally, the polymer forming the jacket 417b can also flow or reflow into the holes 458d of the base 452d. In either case, the base 452d becomes joined, coupled, or mounted to the shaft 411. It will be understood that instead of flowing or reflowing the material forming the jackets 417b, 417d, the base 452d can be mounted to the shaft 411 using adhesives, glues, thermal bonding, laser welding, welding, soldering, mechanical fasteners, such as rivets, detents, threads, brazing, modifications or combinations thereof, crimping, or combinations and modifications thereof.

Extending from a distal end 460d of the base 452d is the tine assembly 454d. The tine assembly 454d includes tines 462d having a generally elongate form. The tines are inclined toward the atraumatic member 456d, with the atraumatic member 456d having a dimension D1 across or transverse to a longitudinal axis L of the tissue encroachment prevention assembly 450d that is smaller than a dimension D2 across or transverse to the longitudinal axis L of the base 452d. This tapered configuration aids with steering the aspiration catheter 402 and provides gaps 464d that allow aspirant and thrombus to pass, while preventing tissue from being drawn into the aspiration lumen 406. Since the tines 462d are inclined towards the atraumatic member 456d, the gaps 464d also form a tapered configuration, however, this need not be case and the gaps 464d can have various shapes and forms to prevent passage of tissue.

Disposed between adjacent tines 462d is a mesh 490d that is proximal to the open space near the high pressure saline spray from supply lumen 414. The mesh 490d provides an additional barrier to prevent vessel tissue from being drawn into the aspiration lumen 406 and into close proximity to the pressurized fluid injection from supply lumen 414. The mesh 490d can include a mesh body 492d with openings 494d that allow blood, clot, or other aspirant to flow into the aspiration lumen 406.

The mesh 490d can be formed at the same time as the tines 462d, such as when the base 452d and tine assembly 454d are cut from a hypotube, for instance. In still another configuration, the base 452d, the mesh 490d, the tines 462d and the shaft 411 are cut from a hypotube, for instance. Alternatively, the mesh 490d can be joined, attached, or otherwise coupled to the base 452d and/or one or more tines 462d such as by welding, laser welding, soldering, gluing, thermal bonding, mechanical attachment, brazing, or combinations or modifications thereof. For instance, when the mesh 490d includes interlaced filaments, wires, threads, etc., a material, such as metallic, polymeric, carbon-fiber, etc. punched, cut, or otherwise formed with the openings 494d, or some other structure, the mesh 490d can be joined, attached, or otherwise coupled to one or more of the base 452d and the tines 462d. In still another configuration, the base 452d can be an extension of the shaft 411 and so the base 452d and the mesh 490d can be formed as part of the shaft 411 to which is attached the tines 462d.

Attached to distal ends 466d of the tines 462d is the atraumatic member 456d. In this particular configuration, the atraumatic member 456d includes a generally spherical ball 468d having an aperture 470d to receive the distal ends 466d, such as illustrated in FIG. 13d. The aperture 470d can be blind hole to receive the distal ends 466d. For instance, when the tissue encroachment prevention assembly 450d is laser cut from a hypotube, each distal end 466d of each tine 462d is disposed in aperture 470d and fixed in place, such as through welding, laser welding, soldering, mechanical fastening, crimping, thermal bonding, gluing, or combination or modifications thereof. While reference is made to an individual aperture 470d receiving the tines 462d, it will be understood that the distal ends 466d of the tines 462d can be received in apertures as described with respect to atraumatic member 456a (FIGS. 9a-d).

As mentioned above, the base 452d (or more generally the tissue encroachment prevention assembly 450d) can replace the radiopaque ring 329 (FIG. 7-8). For example, the base 452d and tines 462d can be formed of stainless steel, with the atraumatic member 456d being made of a radiopaque material, such as a material or alloy containing a material with a higher atomic mass such as tantalum, tungsten, platinum/iridium, gold, silver, and so forth. Alternatively, or in addition to, the atraumatic member 456d being made of a radiopaque material, the base 452d and/or one or more of the tines 462d can be formed of a radiopaque material. In still another configuration, a radiopaque coating can be applied to one or more parts of the tissue encroachment prevention assembly 450d, such as one or more of the base 452d, the tine assembly 454d, the atraumatic member 456d, and/or the mesh 490d.

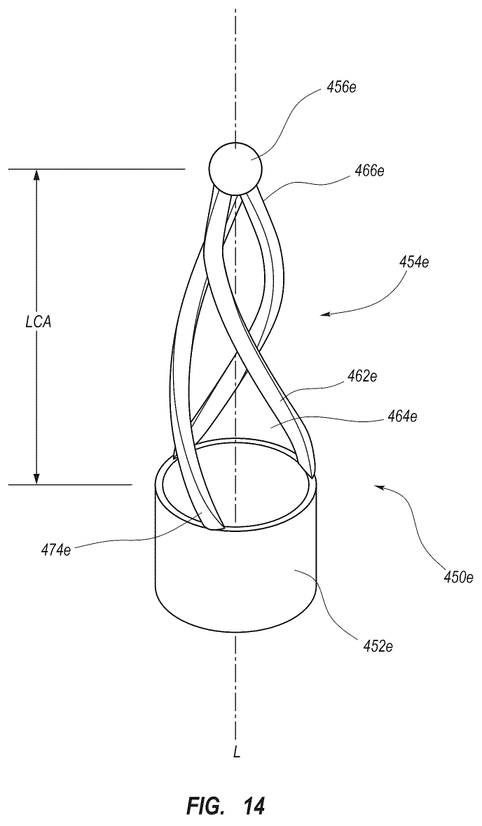

Turning to FIGS. 14-18, illustrated are other tissue encroachment prevention assemblies that provide a barrier to tissue being drawn into an aspiration lumen and thereby control or limit access to the aspiration lumen of the aspiration catheter such that tissue does not encroach or be drawn into the aspiration lumen, and towards the high pressure spray from the supply lumen or one or more supply lumens, while thrombus or aspirant is still capable of being collected. Such tissue encroachment prevention assemblies may provide a means for preventing the blood vessel from being drawn into the aspiration lumen and coming into close proximity to an orifice of a supply lumen (or orifices of one or more supply lumens) during aspiration. Additionally, the tissue encroachment prevention assemblies of FIGS. 14-18 can be advanced into and/or through a clot or thrombus based, in part, upon the particular structure and configuration of the cage assembly. Additional details regarding advancing the tissue encroachment prevention assemblies of FIGS. 14-18 into and/or through a clot or thrombus are provided in detail later.

The tissue encroachment prevention assemblies of FIGS. 14-18 are similar to the tissue encroachment prevention assemblies 450a, 450b, 450c, and 450d and thus the disclosure related to the tissue encroachment prevention assemblies 450a, 450b, 450c, and 450d is also applicable to the description of the tissue encroachment prevention assemblies of FIGS. 14-18. Like reference numerals may be utilized to designate corresponding or similar parts.

Generally, the tissue encroachment prevention assemblies of FIGS. 14-18 can each be mounted to or form part of the aspiration catheters described herein or otherwise contemplated by the disclose contained herein, such as but not limited to aspiration catheters 102, 202, 302, and 402. Therefore, each of the tissue encroachment prevention assemblies of FIGS. 14-18 can be mounted between jackets, such as the jacket 317b or the outer jacket 317d(FIGS. 7-8) or the jacket 417b or the outer jacket 417d (FIG. 9a), and/or be covered by at least one of the jacket 317b, the jacket 317d, the jacket 417b, or the outer jacket 417d (FIGS. 7, 8, 9a). Optionally, the tissue encroachment prevention assemblies of FIGS. 14-18 can be mounted to the shaft 311 or shaft 411, and covered by at least one of the jacket 317b, the outer jacket 317d, the jacket 417b, or the jacket 417d (FIGS. 7, 8, 9a).

As illustrated in FIG. 14, the tissue encroachment prevention assembly 450e includes a base 452e having a generally cylindrical form or ring-like form, a tine assembly 454e extending from the base 452e, and an atraumatic member 456e disposed or arranged at a distal end of the tissue encroachment prevention assembly 450e. The combination of the base 452e, the tine assembly 454e, and the atraumatic member 456e forms a cage-like structure that allows clot, thrombus, aspirant, blood, etc. to flow into the aspiration lumen and prevent vessel tissue from being drawing into the aspiration lumen 406. The tines 462e of the tine assembly 454e extend from the base 452e to the atraumatic member 456e in a spiraling, twisting, or helical fashion about and toward a longitudinal axis L of the tissue encroachment prevention assembly 450e so that a proximal end 474e of each tine 462e is farther from the longitudinal axis L, in a direction transverse to the longitudinal axis L, than a distal end 466e of each tine 462e. This results in the tine assembly 454e having a multi-tine corkscrew shape having gaps 464e through which clots or aspirants can be drawn towards an aspiration lumen, while preventing the vessel tissue from being drawn into close proximity to high pressure saline spray from the supply lumen. The tines 462e have a generally elongate form with a generally rectangular cross-sectional (although circular, oval, square, polygonal, and combinations or modifications thereof are also possible).

The tines 462e can complete one turn, less than one turn, or multiple turns between the base 452e and the atraumatic member 456e. A tine 462e extends “one turn” as it spirals, twists, or extends in a helical fashion about 360 degrees around the longitudinal axis L. As illustrated in FIG. 14, each tine 462e rotates about 115 degrees to about 125 degrees over a length LCA between the base 452e and the atraumatic member 456e. In other configuration, over a length LCA each tine 462e rotates about 15 degrees to about 180 degrees, about 180 degrees to about 360 degrees, about 360 degrees to about 720 degrees, about 720 degrees to about 1080 degrees, about 115 degrees to about 1080 degrees, or is in a range between any two of the foregoing. The length LCA ranges from about 5 mm to about 6 mm, from about 6 mm to about 10 mm, from about 10 mm to about 20 mm, from about 20 mm to about 25 mm, from about 5 mm to about 25 mm, or is in a range between any two of the foregoing. Stated another way, a length of the tines can range from about 0.2″ to about 0.24″, from about 0.24″ to about 0.39″, from about 0.39″ to about 0.79″, from about 0.79″ to about 0.98″, from about 0.2″ to about 0.98″ mm, or is in a range between any two of the foregoing. The base 452e, the tines 462e, and/or the atraumatic member 456e is formed of a metal, alloy, shape-memory material, composite, polymer, or combinations or modifications thereof.

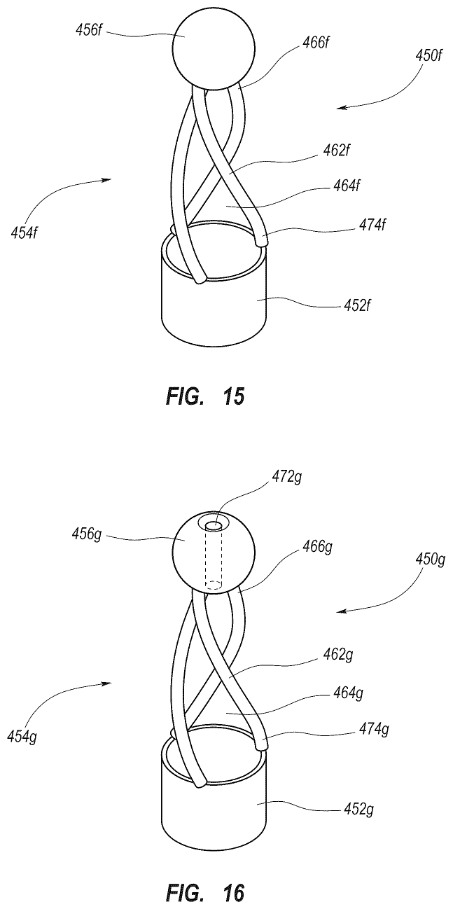

As illustrated in FIG. 15, the tissue encroachment prevention assembly 450f includes a base 452f having a generally cylindrical form or ring-like form, a tine assembly 454f extending from the base 452f, and an atraumatic member 456f disposed or arranged at a distal end of the tissue encroachment prevention assembly 450f. The combination of the base 425f, the tine assembly 454f, and the atraumatic member 456f forms a cage-like structure that allows clot, thrombus, aspirant, blood, etc. to flow into the aspiration lumen and prevent vessel tissue from being drawing into the aspiration lumen 406. The tines 462f of the tine assembly 454f extend from the base 452f to the atraumatic member 456f in a spiraling, twisting, or helical fashion about and toward a longitudinal axis L of the tissue encroachment prevention assembly 450f so that a proximal end 474f of each tine 462f is farther from the longitudinal axis L, in a direction transverse to the longitudinal axis L, than a distal end 466f of each tine 462f. This results in the tine assembly 454f having a multi-tine corkscrew shape having gaps 464f through which clots or aspirants can be drawn towards an aspiration lumen, while preventing the vessel tissue from being drawn into close proximity to a high pressure saline spray from the supply lumen. The tines 462f have a generally elongate form with a generally circulate cross-sectional (although, oval, square, polygonal, and combinations or modifications thereof are also possible).

The tines 462f can complete one turn, less than one turn, or multiple turns between the base 452f and the 456f. A tine 462f extends “one turn” as it spirals, twists, or extends in a helical fashion about 360 degrees around the longitudinal axis L. As illustrated in FIG. 15, each tine 462f rotates about 115 degrees to about 125 degrees over a length LCA between the base 452f and the atraumatic member 456f. In other configuration, over a length LCA each tine 462f rotates about 15 degrees to about 180 degrees, about 180 degrees to about 360 degrees, about 360 degrees to about 720 degrees, about 720 degrees to about 1080 degrees, about 115 degrees to about 1080 degrees, or is in a range between any two of the foregoing. The length LCA ranges from about 5 mm to about 6 mm, from about 6 mm to about 10 mm, from about 10 mm to about 20 mm, from about 20 mm to about 25 mm, from about 5 mm to about 25 mm, or is in a range between any two of the foregoing. Stated another way, a length of the tines can range from about 0.2″ to about 0.24″, from about 0.24″ to about 0.39″, from about 0.39″ to about 0.79″, from about 0.79″ to about 0.98″, from about 0.2″ to about 0.98″ mm, or is in a range between any two of the foregoing. The base 452f, the tines 462f, and/or the atraumatic member 456f is formed of a metal, alloy, shape-memory material, composite, polymer, or combinations or modifications thereof.

As illustrated in FIG. 16, a tissue encroachment prevention assembly 450g includes a base 452g having a generally cylindrical form or ring-like form, a tine assembly 454g extending from the base 452g, and an atraumatic member 456g disposed or arranged at a distal end of the tissue encroachment prevention assembly 450g. The combination of the base 425g, the tine assembly 454g, and the atraumatic member 456g forms a cage-like structure that allows clot, thrombus, aspirant, blood, etc. to flow into the aspiration lumen 406 while preventing vessel tissue from being drawing into the aspiration lumen 406. The tines 462g of the tine assembly 454g extend from the base 452g to the atraumatic member 456g in a spiraling, twisting, or helical fashion about and toward a longitudinal axis L of the tissue encroachment prevention assembly 450g so that a proximal end 474g of each tine 462g is farther from the longitudinal axis L, in a direction transverse to the longitudinal axis L, than a distal end 466g of each tine 462g. This results in the tine assembly 454g having a multi-tine corkscrew shape having gaps 464g through which clots or aspirants can be drawn towards an aspiration lumen 406, while preventing the vessel tissue from being drawn into close proximity to a high pressure saline spray from the supply lumen. The tines 462g have a generally elongate form with a generally rectangular cross-sectional (although circular, oval, square, polygonal, and combinations or modifications thereof are also possible).

The tines 462g can complete one turn, less than one turn, or multiple turns between the base 452g and the 456g. A tine 462g extends “one turn” as it spirals, twists, or extends in a helical fashion about 360 degrees around the longitudinal axis L. As illustrated in FIG. 16, each tine 462g rotates about 115 degrees to about 125 degrees over a length LCA between the base 452g and the atraumatic member 456f. In other configuration, over a length LCA each tine 462g rotates about 15 degrees to about 180 degrees, about 180 degrees to about 360 degrees, about 360 degrees to about 720 degrees, about 720 degrees to about 1080 degrees, about 115 degrees to about 1080 degrees, or is in a range between any two of the foregoing. The length LCA ranges from about 5 mm to about 6 mm, from about 6 mm to about 10 mm, from about 10 mm to about 20 mm, from about 20 mm to about 25 mm, from about 5 mm to about 25 mm, or is in a range between any two of the foregoing. Stated another way, a length of the tines can range from about 0.2″ to about 0.24″, from about 0.24″ to about 0.39″, from about 0.39″ to about 0.79″, from about 0.79″ to about 0.98″, from about 0.2″ to about 0.98″ mm, or is in a range between any two of the foregoing. The base 452g, the tines 462g, and/or the atraumatic member 456g is formed of a metal, alloy, shape-memory material, composite, polymer, or combinations or modifications thereof.

Formed in the atraumatic member 456g is a through-hole 472g that can accommodate a guidewire or other structure to aid with guiding an aspiration catheter to clot or thrombus to be removed. The through-hole 472g can include chamfers or other structures to guide the guidewire, for instance, through the through-hole 472g. The through-hole 472g can accommodate a 0.014″ guidewire. More generally, each through-hole 472g can have a diameter ranging from about 0.36 mm to about 0.51 mm, from about 0.51 mm to about 0.61 mm, from about 0.61 mm to about 0.71 mm, from about 0.71 mm to about 0.81 mm, from about 0.36 mm to about 0.81 mm, or is in a range between any two of the foregoing. Stated another way, each through-hole 472g can have a diameter ranging from about 0.01″ to about 0.02″, from about 0.02″ to about 0.024″, from about 0.024″ to about 0.028″, from about 0.028″ to about 0.032″, from about 0.01″ to about 0.032″, or is in a range between any two of the foregoing. Additionally, each through-hole 472g of the atraumatic member 456g can have the same or different size and/or shape.

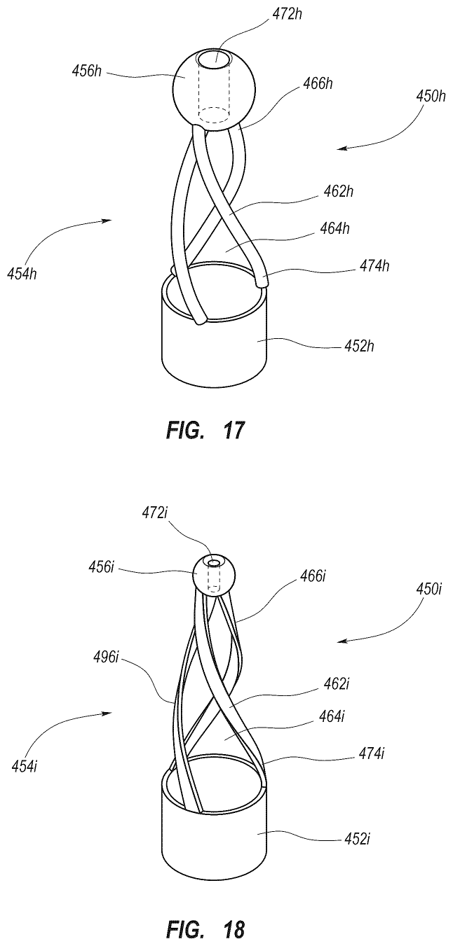

FIG. 17 illustrates a tissue encroachment prevention assembly 450h similar to tissue encroachment prevention assembly 450g, except that the through-hole 472h can accommodate a 0.036″ guidewire. As such, the through-hole 472h in the atraumatic member 456h, which is mounted to a tine assembly 454h that is mounted to a base 452h, can have a diameter ranging from about 0.91 mm to about 1.02 mm, from about 1.02 mm to about 1.12 mm, from about 1.12 mm to about 1.22 mm, from about 1.22 mm to about 1.32 mm, from about 0.9 mm to about 2 mm, or is in a range between any two of the foregoing. Stated another way, the through-hole 472h in the atraumatic member 456h can have a diameter ranging from about 0.036″ to about 0.04″, from about 0.04″ to about 0.044″, from about 0.044″ to about 0.048″, from about 0.048″ to about 0.052″, from about 0.036″ to about 0.079″, or is in a range between any two of the foregoing.

As illustrated in FIG. 18, the tissue encroachment prevention assembly 450i includes a base 452i having a generally cylindrical form or ring-like form, a tine assembly 454i extending from the base 452i, and an atraumatic member 456i disposed or arranged at a distal end of the tissue encroachment prevention assembly 450i. The combination of the base 452i, the tine assembly 454i, and the atraumatic member 456i forming a cage-like structure that allows clot, thrombus, aspirant, blood, etc. to flow into the aspiration lumen and prevent vessel tissue from being drawing into the aspiration lumen 406. The tines 462i of the tine assembly 454i extend from the base 452i to the atraumatic member 456i in a spiraling, twisting, or helical fashion about and toward a longitudinal axis L of the tissue encroachment prevention assembly 450i so that a proximal end 474i of each tine 462i is farther from the longitudinal axis L, in a direction transverse to the longitudinal axis L, than a distal end 466i of each tine 462i. This results in the tine assembly 454i having a multi-tine corkscrew shape having gaps 464i through which clots or aspirants can be drawn towards an aspiration lumen, while preventing the vessel tissue from being drawn into close proximity to a high pressure saline spray from the supply lumen. The tines 462i have a generally elongate form with a generally rectangular cross-sectional (although circular, oval, square, polygonal, and combinations or modifications thereof are also possible). The tines 462i have a ribbon-like form having edges 496i that can cut thrombus as the tissue encroachment prevention assembly 450i is advanced through thrombus. The edges 496i can optionally be sharpened to enhance the cutting action or capabilities of the tines 462i. While edges 496i are illustrated in the configuration of FIG. 18, it will be understood that edges, cutting edges, or sharpened edges can be formed on any of the tines of any of the tissue encroachment prevention assemblies of the present disclosure.

The tines 462i can complete one turn, less than one turn, or multiple turns between the base 452i and the 456i. A tine 462i extends “one turn” as it spirals, twists, or extends in a helical fashion about 360 degrees around the longitudinal axis L. As illustrated in FIG. 18, each tine 462i rotates about 115 degrees to about 125 degrees over a length LCA between the base 452i and the atraumatic member 456i. In other configuration, over a length LCA each tine 462i rotates about 15 degrees to about 180 degrees, about 180 degrees to about 360 degrees, about 360 degrees to about 720 degrees, about 720 degrees to about 1080 degrees, about 115 degrees to about 1080 degrees, or is in a range between any two of the foregoing. The length LCA ranges from about 5 mm to about 6 mm, from about 6 mm to about 10 mm, from about 10 mm to about 20 mm, from about 20 mm to about 25 mm, from about 5 mm to about 25 mm, or is in a range between any two of the foregoing. Stated another way, a length of the tines can range from about 0.2″ to about 0.24″, from about 0.24″ to about 0.39″, from about 0.39″ to about 0.79″, from about 0.79″ to about 0.98″, from about 0.2″ to about 0.98″ mm, or is in a range between any two of the foregoing. The base 452i, the tines 462i, and/or the atraumatic member 456i is formed of a metal, alloy, shape-memory material, composite, polymer, or combinations or modifications thereof.

As mentioned above, the tissue encroachment prevention assemblies of FIGS. 14-18 can be advanced into and/or through a clot or thrombus such as by using sliding movement, rotation movement, and/or a combination of sliding and rotational movement. For instance, with the tissue encroachment prevention assembly disposed at a distal end of the shaft of the aspiration catheter, sliding and/or rotating the shaft rotates the tissue encroachment prevention assembly and thereby screws the tissue encroachment prevention assembly into and/or through clot or thrombus. This action can also break up the clot or thrombus to aid with aspiration of the clot or thrombus.

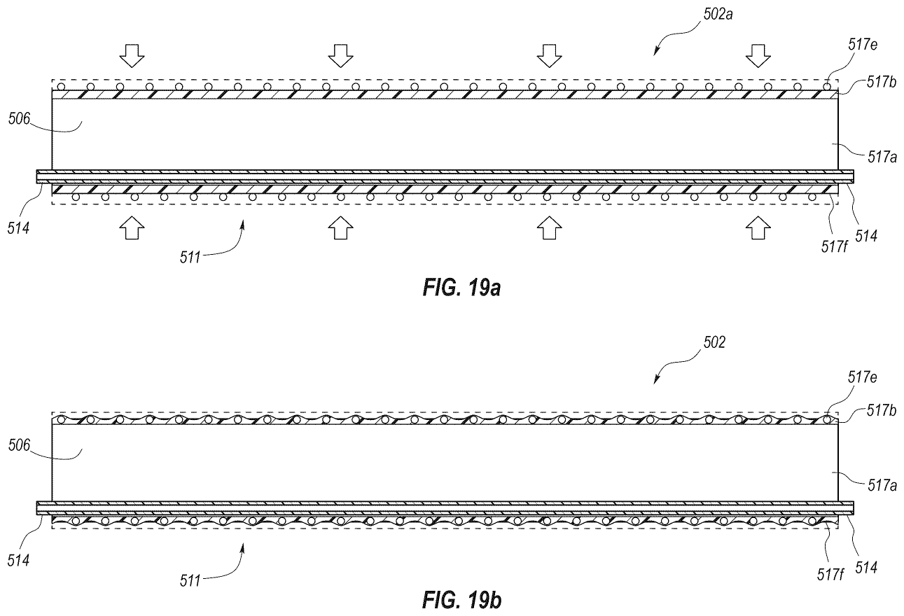

Turning to FIGS. 19a and 19b, illustrated is one configuration of an aspiration catheter 502a that can be rotated to advance a tissue encroachment prevention assembly, such as those described in relation to FIGS. 14-18, and any of the other tissue encroachment prevention assemblies described herein. Such tissue encroachment prevention assembly may provide a means for preventing the blood vessel from being drawn into the aspiration lumen and coming into close proximity to an orifice of a supply lumen during aspiration. The aspiration catheter 502a can be any of the aspiration catheters described herein, such as aspiration catheters 102, 202, 302, and 402, and so the disclosure related to aspiration catheters 102, 202, 302, and 402 is also applicable to the aspiration catheter 502a.

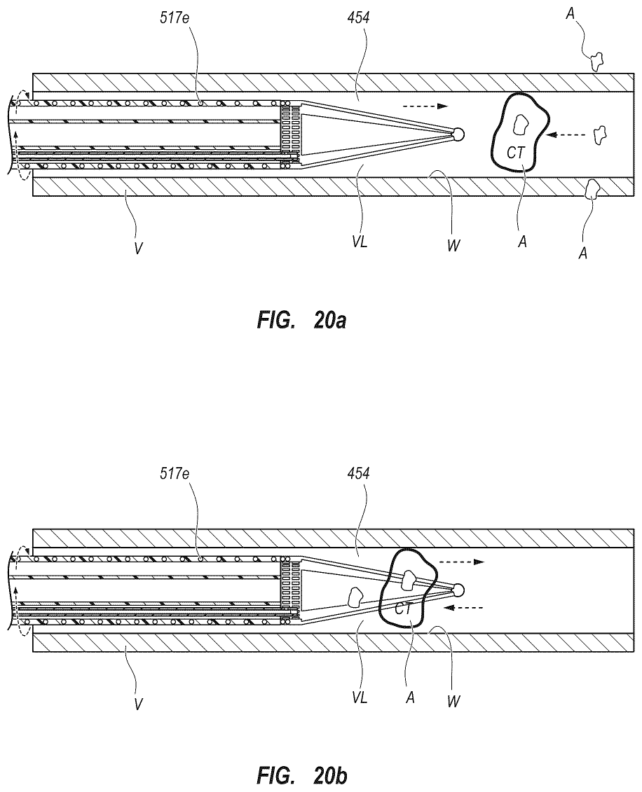

As illustrated in FIG. 19a, which is a partially deconstructed illustration of a portion of the aspiration catheter 502a, the aspiration catheter 502a includes an aspiration lumen 506 formed by a shaft 511, such as a hypotube. A supply lumen 514 is disposed within the aspiration lumen 506 and can be configured to provide a high pressure fluid injection, such as saline, within the aspiration lumen 506 for macerating a thrombus as it is aspirated, such as illustrated in FIGS. 20a-20b. The shaft 511 can be advanced within the vessel V with the aid of a screw or threaded structure. In the illustrated configuration, the screw structure combines a jacket 517b that at least partially covers a shaft body 517a of the shaft 511 and a screw member 517e that is attached to or formed with the jacket 517b. For instance, as illustrated in FIG. 19a, the screw member 517e is separate from the jacket 517b and then brought into contact and heated with the jacket 517b so that the screw member 517e and the jacket 517b will melt together. Alternatively, or in addition to the above, the jacket 517b and the screw member 517e can be brought into contact with one another and a shrink tube 517f applied around the jacket 517b and the screw member 517e. When heated, the shrink tube 517f will reduce its diameter, pressing the screw member 517e against the jacket 517b. The heat will also melt the screw member 517e and the jacket 517b forming a monolithic, unitary structure. In still another configuration, the screw member 517e can be applied directly to the shaft body 517a and the shrink tube 517f applied around the screw member 517e and the shaft body 517a. As discussed above, when heated, the shrink tube 517f will reduce its diameter, pressing the screw member 517e against the shaft body 517a.

In positioning the screw member 517e, a direction of wrapping the screw member 517e around the shaft body 517a, and/or the jacket 517b, is the same as a screw direction of the tissue encroachment prevention assembly, i.e., a turn direction of the tines 462 of the tine assemblies 454. This allows rotation of the shaft, aided by the screw member, to rotate the tissue encroachment prevention assembly in a direction that screws the tissue encroachment prevention assembly into and/or through the clot or thrombus CT, such as illustrated in FIGS. 20a-20b. The screwing action can also engage cutting edges (e.g., edges 496i of FIG. 18) with the clot or thrombus CT to help separate or break up the clot or thrombus CT, thereby making it easier to aspirate the clot or thrombus. Reversing the direction of rotation can remove the tine assembly 454 from within the clot or thrombus. A combination of forward and reverse movement can aid with removing the clot or thrombus. Additionally, materials and geometry of the tines can be selected to change the size or geometry of the gaps 464 of the tine assembly 454, such as illustrated in phantom in FIG. 20b. For instance, when the tines are formed to have increased elasticity, ability to deform or move, or include preferential bending regions (i.e., cuts, thinned dimension as compared to other portions of the tine, etc.), the tine assembly 454 can partially collapse or change shape to increase a size of the gap 464. This change allows larger portions of the clot or thrombus to extend into and through the gaps 464, optionally extending into the aspiration lumen, and be available for aspiration through the aspiration lumen. The bulbous or mushroom type shape also maintains the vessel wall W at a distance from the aspiration lumen to prevent portions of the vessel wall W from being drawn into the aspiration lumen.

The jacket 517b, the screw member 517e, and shrink tube 517f can be formed of various types of polymeric material. For instance, the jacket 517b, the screw member 517e, and shrink tube 517f can be formed of PEBAX, polytetrafluoroethylene (PTFE), polyethylene terephthalate (PET) combinations or modifications thereof. Additionally, the shrink tube 517f can be formed of a low friction polytetrafluoroethylene (PTFE) tubing. Optionally, the shrink tube 517f can have striping, spiral, etc. of an of the proceeding.

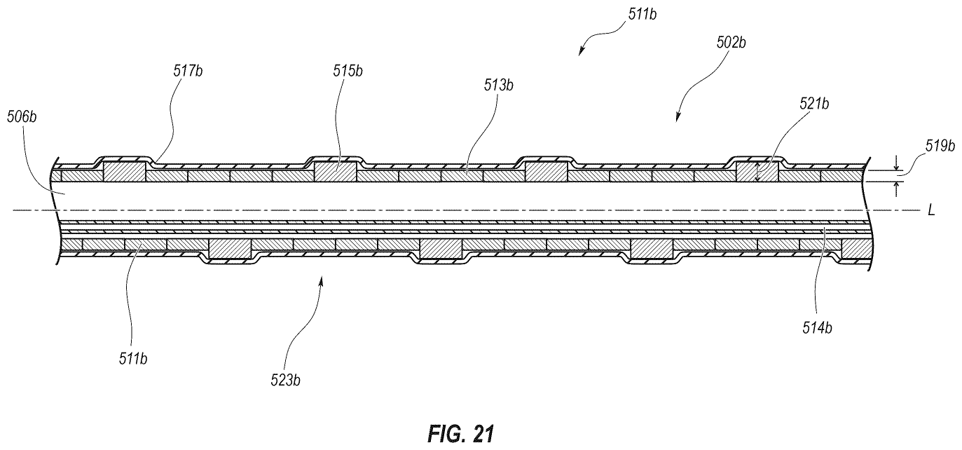

Turning to FIG. 21, illustrated is another aspiration catheter 502b that can be rotated to advance a tissue encroachment prevention assembly, such as those described in relation to FIGS. 14-18, and any of the other tissue encroachment prevention assemblies described herein. Such tissue encroachment prevention assembly may provide a means for preventing the blood vessel from being drawn into the aspiration lumen and coming into close proximity to an orifice of a supply lumen (or orifices of one or more supply lumens) during aspiration. The aspiration catheter 502b can be any of the aspiration catheters described herein, such as aspiration catheters 102, 202, 302, 402, and 502a and so the disclosure related to aspiration catheters 102, 202, 302, 402, and 502a is also applicable to the aspiration catheter 502b.

As illustrated in FIG. 21, the aspiration catheter 502b includes a shaft 511b forming an aspiration lumen 506b. A supply lumen 514b is disposed within the aspiration lumen 506b and can be configured to provide a high pressure fluid injection, such as saline, within the aspiration lumen 506b for macerating a thrombus as it is aspirated. The shaft 511b is formed from a first coil member 513b and a second coil member 515b, with a shrink tube 517f formed around the first coil member 513b and the second coil member 515b. The first coil member 513b has a first wall thickness 519b, while the second coil member 515b has a second wall thickness 521b larger than the first wall thickness 519b. In this way, an outer surface 523b of the shaft 511b provides a “screw”- or “step”-like structure with peak portions (from the second coil member 515b) and valley portion (formed by the first coil member 513b between adjacent second coil members 515b). As shrink tubing 517f is formed around and seals the first coil member 513b and the second coil member 515b, the screw or thread structure for the shaft 511b is formed.

To form the shaft 511b, the first coil member 513b is stretched along the longitudinal axis L to provide a series of gaps into which the second coil member 515b is wrapped or disposed. When the longitudinal force is released, the second coil members 515b are sandwiched between coils of the first coil member 513b. The second coil member 515b can be held by the force applied by the first coil member 513b and/or the shrink tube 517f. Additionally, or alternatively, the second coil member 515b can be welded, such as by laser welding, or otherwise attached to the first coil member 513b to provide better resistance against longitudinal stretching of the shaft 511b. In another configuration, the first coil member 513b and the second coil member 515b are wrapped together around a mandrel to form the shaft 511b. The mandrel maintains the inside diameter of the aspiration lumen 506b, while the differences in wall thicknesses, i.e., first wall thickness 519b and the second wall thickness 521b, create the stepped or screw outer surface 523b. After jacketing the first coil member 513b and the second coil member 515b with the shrink tube 517f, the shaft 511b shows a helical outer structure, i.e., the outer surface 523b, which will help the aspiration catheter 502b to advance forward when turned into the direction of the helix and, therefore, will better engage with the thrombus.

As described in relation to aspiration catheter 502a, forming the shaft 511b, the first coil member 513b and the second coil member 515b are wrapped in the same direction as a screw direction of the tissue encroachment prevention assembly, i.e., a turn direction of the tines 462 of the tine assembly 454. This allows rotation of the shaft, aided by the screw member, to rotate the tissue encroachment prevention assembly in a direction that screws the tissue encroachment prevention assembly into and/or through the clot or thrombus CT. The screwing action can also engage cutting edges (e.g., edges 496i of FIG. 18) with the clot or thrombus CT to help separate or break up the clot or thrombus CT, thereby making it easier to aspirate the clot or thrombus. Reversing the direction of rotation can remove the tine assembly 454 from within the clot or thrombus. A combination of forward and reverse movement can aid with removing the clot or thrombus.

The first coil member 513b and the second coil member 515b can be formed of wires or members having a variety of different cross-sectional shapes. A cross-sectional shape of the first coil member 513b and the second coil member 515b can be round, oval, polygonal, square, rectangular, or combinations or modifications thereof. A cross-sectional shape of the first coil member 513b can be different from that of the second coil member 515b, such as in size, shape, etc.

The first coil member 513b and the second coil member 515b can be formed of a variety of different materials, such as but not limited to metals, alloys, polymers, composites, combinations of modifications thereof. A material forming the first coil member 513b can be different from that of the second coil member 515b.

The shrink tube 517f can be formed of various types of polymeric material. For instance, the shrink tube 517f can be formed of low friction polytetrafluoroethylene (PTFE), PEBAX, polyethylene terephthalate (PET), fluorinated ethylene propylene (FEP), combinations or modifications thereof. Optionally, the shrink tube 517f can have striping, spiral, etc. of an of the proceeding.

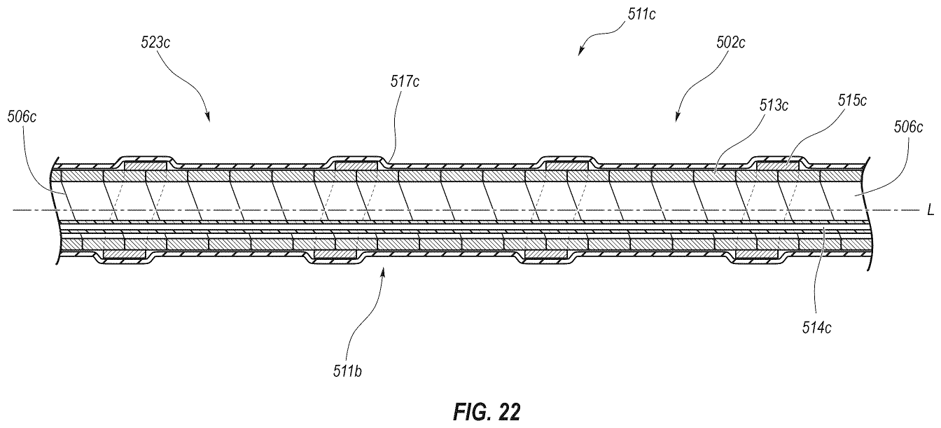

Turning to FIG. 22, illustrated is another aspiration catheter 502c that can be rotated to advance a tissue encroachment prevention assembly, such as those described in relation to FIGS. 14-18, and any of the other tissue encroachment prevention assemblies described herein. Such tissue encroachment prevention assembly may provide a means for preventing the blood vessel from being drawn into the aspiration lumen and coming into close proximity to an orifice of a supply lumen (or orifices of one or more supply lumens) during aspiration. The aspiration catheter 502c can be any of the aspiration catheters described herein, such as aspiration catheters 102, 202, 302, 402, 502a, and 502b and so the disclosure related to aspiration catheters 102, 202, 302, 402, 502a, 502c is also applicable to the aspiration catheter 502c.

As illustrated in FIG. 22, the aspiration catheter 502c includes a shaft 511c forming an aspiration lumen 506c. A supply lumen 514c is disposed within the aspiration lumen 506c and can be configured to provide a high pressure fluid injection, such as saline, within the aspiration lumen 506c for macerating a thrombus as it is aspirated. The shaft 511c is formed from a first coil member 513c and a second coil member 515c, with a shrink tube 517c formed around the first coil member 513c and the second coil member 515c. Unlike the shaft 511b of the aspiration catheter 502b, the second coil member 515c is formed on the first coil member 513c to form an outer surface 523c of the shaft 511c with a “screw”- or “step”-like structure with peak portions (from the second coil member 515c) and valley portion (formed by the first coil member 513c between adjacent second coil members 515c). For instance, the second coil member 515c is wrapped around/on the first coil member 513c and the shrink tube 517f is heated so that it decreases in diameter to form the outer surface 523c. The second coil member 515c extending from a surface of the first coil member 513c creates the stepped or screw outer surface 523c.

As described in relation to aspiration catheter 502a, forming the shaft 511c, the second coil member 515c is wrapped in the same direction as a screw direction of the tissue encroachment prevention assembly, i.e., a turn direction of the tines 462 of the tine assembly 454, with the first coil member 513c being wrapped in the opposite direction. This allows rotation of the shaft, aided by the screw member, to rotate the tissue encroachment prevention assembly in a direction that screws the tissue encroachment prevention assembly into and/or through the clot or thrombus CT. The screwing action can also engage cutting edges (e.g., edges 496i of FIG. 18) with the clot or thrombus CT to help separate or break up the clot or thrombus CT, thereby making it easier to aspirate the clot or thrombus. Reversing the direction of rotation can remove the tine assembly 454 from within the clot or thrombus. A combination of forward and reverse movement can aid with removing the clot or thrombus.

The second coil member 515c being wrapped in an opposite direction to the first coil member 513c provides better resistance against longitudinal stretching of the shaft 511c. The degree of stretching can also be varied by changing a pitch of the second coil member 515c to provide more close or more open form to the second coil member 515c. After jacketing the first coil member 513c and the second coil member 515c with the shrink tube 517f, the shaft 511c shows a helical outer structure, i.e., the outer surface 523c, which will help the aspiration catheter 502c to advance forward when turned into the direction of the helix and, therefore, will better engage with the thrombus.

The first coil member 513c and the second coil member 515c can be formed of wires or members having a variety of different cross-sectional shapes. A cross-sectional shape of the first coil member 513c and the second coil member 515c can be round, oval, polygonal, square, rectangular, or combinations or modifications thereof. A cross-sectional shape of the first coil member 513c can be different from that of the second coil member 515c, such as in size, shape, etc.

The first coil member 513c and the second coil member 515c can be formed of a variety of different materials, such as but not limited to metals, alloys, polymers, composites, combinations of modifications thereof. A material forming the first coil member 513c can be different from that of the second coil member 515c.

The shrink tube 517f can be formed of various types of polymeric material. For instance, the shrink tube 517f can be formed of low friction polytetrafluoroethylene (PTFE), PEBAX, polyethylene terephthalate (PET), fluorinated ethylene propylene (FEP), combinations or modifications thereof. Optionally, the shrink tube 517f can have striping, spiral, etc. of an of the proceeding.

As mentioned above, during aspiration, damage to the vessel might occur because the vessel wall is drawn into the aspiration lumen and comes into contact with the pressurized fluid injection from a supply lumen. The tissue encroachment prevention assemblies disclosed herein prevent an aspiration catheter from damaging the vessel during aspiration. The tissue encroachment prevention assemblies described thus far are fixed to an end of the aspiration catheter and extend distally from a distal end of the aspiration catheter in a pre-deployment state as the aspiration catheter is moved through the tortuous patient anatomy, during deployment of the aspiration catheter to aspirate clot or thrombus, and during withdrawal of the aspiration catheter following completion of clot or thrombus removal. While having a fixed tissue encroachment prevention assembly extending distally from an end of the aspiration catheter is one configuration, in other configurations, a deployable and retractable tissue encroachment prevention assembly is also contemplated. The deployable and retractable tissue encroachment prevention assembly simplifies passage through the tortuous patient anatomy, while preventing vessel tissue from being drawn into an aspiration lumen of the catheter or toward or into close proximity to a high pressure saline spray from a supply lumen and associated orifice.

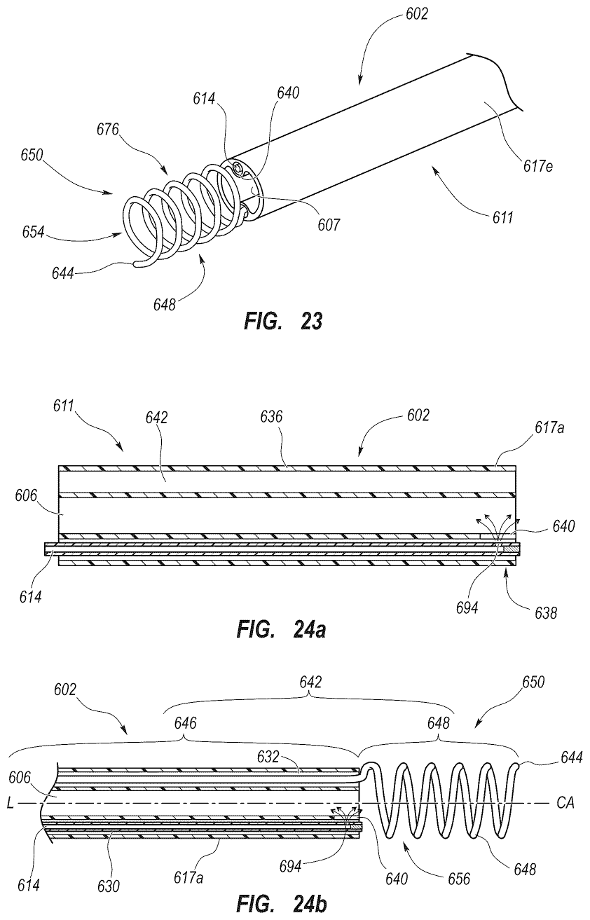

Turning to FIGS. 23-26b, illustrated is an aspiration catheter 602 having a tissue encroachment prevention assembly 650 that is deployable and retractable. Such tissue encroachment prevention assembly may provide a means for preventing the blood vessel from being drawn into the aspiration lumen and coming into close proximity to an orifice of a supply lumen during aspiration. The aspiration catheter 602 can be any of the aspiration catheters described herein, such as aspiration catheters 102, 202, 302, 402, 502a, 502b, and 502c and so the disclosure related to aspiration catheters 102, 202, 302, 402, 502a, 502bc, and 502c is also applicable to the aspiration catheter 602. Additionally, the disclosure related to the other tissue encroachment prevention assemblies is also applicable to the tissue encroachment prevention assembly 650.

As illustrated in FIG. 23, the aspiration catheter 602 includes a shaft 611 possessing three lumens, such as an aspiration lumen 606, a supply tube lumen 630 that receives a supply tube with the supply lumen 614, and a tissue encroachment prevention assembly lumen 632 that receives the tissue encroachment prevention assembly 650. The shaft 611 can be a multi-lumen extruded tube, such as a tube formed of a polymer, PEBAX, VESTAMID, nylon, polyurethane, combinations or modifications thereof, and optionally reinforced with braids, wires, etc. Alternatively, the shaft 611 can be a hypotube or other tubular member with tubular members disposed within the shaft 611 to form the supply tube lumen 630 and the tissue encroachment prevention assembly 650. Stated another way, the supply tube lumen 630 and the tissue encroachment prevention assembly 650 can be formed of tubular members that are disposed within the aspiration lumen 606 of the shaft 611.

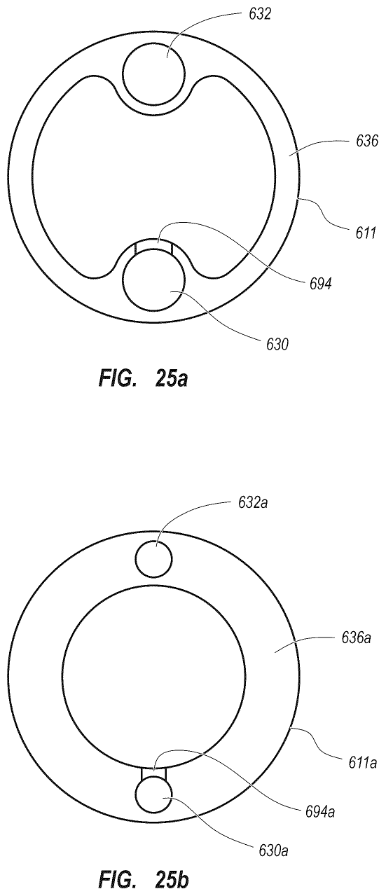

The aspiration lumen 606 is formed towards a center of the shaft 611, such as illustrated in FIGS. 23 and 25a, with the supply tube lumen 630 and the tissue encroachment prevention assembly lumen 632 formed in a wall 636 of a shaft body 617a of the shaft 611. As illustrated in FIGS. 25a, for instance, the wall 636 protrudes inwardly towards a center of the aspiration lumen 606 to accommodate the supply tube lumen 630 and the tissue encroachment prevention assembly lumen 632. However, as illustrated in FIG. 25b, in other configurations, the wall 636a does not protrude inwardly towards the center to accommodate the supply tube lumen 630 and the tissue encroachment prevention assembly lumen 632.

With continued reference to FIGS. 23-25a, disposed in the wall 636 towards a distal end 638 of the supply tube lumen 630 is an opening 640 that is aligned with an orifice 694 for ejection or jetting fluid into the aspiration lumen 606. The supply lumen 614 is connected to or fluidly coupled to a high pressure fluid source (e.g., SDU 212) to provide the fluid. This fluid traverses the aspiration lumen 606 to macerate or break up the thrombus, clot, aspirant, etc. that is drawn into the aspiration lumen 606.

Disposed within the tissue encroachment prevention assembly lumen 632 is the tissue encroachment prevention assembly 650. The tissue encroachment prevention assembly 650 can be advanced from within the tissue encroachment prevention assembly lumen 632 through movement of a handle or other actuator disposed at the handpiece 233 (FIG. 4). The tissue encroachment prevention assembly 650 includes an elongate member 642 with an atraumatic member 644 including a portion that transitions from a first state when retained within the tissue encroachment prevention assembly lumen 632 (see FIG. 24a) to a second state when the portion of the elongate member 642 is disposed from a distal end of the tissue encroachment prevention assembly lumen 632 (see FIG. 24b). A first portion 646 of the elongate member 642 maintains the same shape or state inside or outside of the tissue encroachment prevention assembly lumen 632, while a second portion 648 of the elongate member 642, which includes a tissue encroachment prevention member 676, transitions to a second state in which the tissue encroachment prevention member 676 will prevent the vessel tissue from being drawn into close proximity to a high pressure saline spray from the supply lumen 614. As the tissue encroachment prevention member 676 transitions to the second state, the rotational motion to the coiled state can draw the aspiration catheter 602 towards the clot or thrombus as the tissue encroachment prevention member 676 enters the clot or thrombus. The tissue encroachment prevention member 676 transitions to a coiled state having a coil axis coincident with a longitudinal axis L of the aspiration catheter 602 such that a channel 654 formed by the tissue encroachment prevention member 676 is aligned with the aspiration lumen 606 to aid with passage of clot or thrombus. In other configurations, however, the coil axis CA of the coiled state of the tissue encroachment prevention member 676, and/or the channel 654, are not coincident with the longitudinal axis L of the aspiration lumen 606 but are offset from, and optionally parallel or transverse to, the longitudinal axis L.

The elongate member 642 can be a wire, a hollow tube, a rod, combinations or modifications thereof that is formed to return to a pre-defined shape, i.e., the coiled state, following advancing the tissue encroachment prevention member 676 from the elongate member 642. For instance, the tissue encroachment prevention member 676, and the elongate member 642 as a whole, can be formed of a shape memory material, such as a shape member alloy or metal or a shape member polymer, a spring steel or alloy, combinations of modifications thereof. Alternatively, the tissue encroachment prevention member 676 can be formed of a material, or otherwise worked to form the pre-defined shape, while a reminder of the elongate member 642, such as first portion 646, is formed of a different material. For instance, the first portion 646 can be formed of stainless steel, while the tissue encroachment prevention member 676 is formed of Nitinol.

A pitch and number of the coils also can be varied to increase or decrease a flexibility of the tissue encroachment prevention member 676. The pitch can range from about 1 mm to about 2 mm, from about 2 mm to about 4 mm, from about 4 mm to about 6 mm, from about 6 mm to about 10 mm, from about 1 mm to about 10 mm, from about 2 mm to about 8 mm, or is in a range between any two of the foregoing. Stated another way, the pitch can range from about 0.039″ to about 0.079″, from about 0.079″ to about 0.157″, from about 0.157″ to about 0.236″, from about 0.236″ to about 0.394″, from about 0.039″ to about 0.394″, from about 0.079″ to about 0.315″, or is in a range between any two of the foregoing. The number of turns or coils associated with the tissue encroachment prevention member 676 can range from about 2 to about 4 from about 4 to about 6, from about 6 to about 12, from about 2 to about 12, or is in a range between any two of the foregoing. The material selected for the tissue encroachment prevention member 676 can also aid with varying a resiliency, flexibility, spring force, and elasticity of the tissue encroachment prevention member 676 and its ability to rebound or move the distal end of the aspiration catheter 602 from a tissue wall.

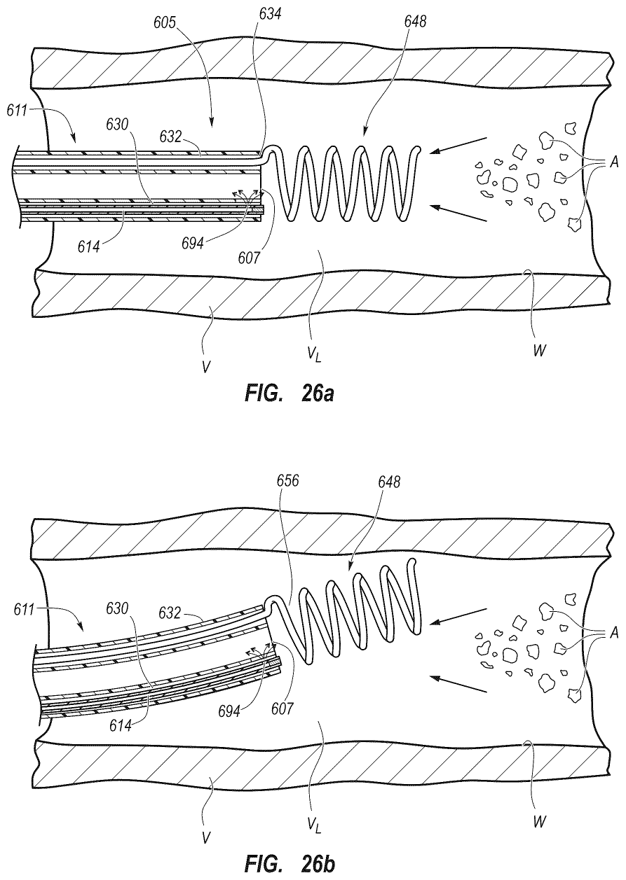

Turning to FIGS. 26a and 26b, the tissue encroachment prevention assembly 650 is illustrated preventing tissue from being drawn into the aspiration lumen 606. As the aspiration catheter 602 is moved to a position to aspirate a clot, thrombus, or aspirant A, a clinician can advance the tissue encroachment prevention member 676 from the tissue encroachment prevention assembly lumen 632 and from the distal opening 634. As the tissue encroachment prevention member 676 exits the distal opening 634, it returns to the pre-set or pre-formed shape extending distally from the distal end 605 of the aspiration catheter 602. When the tissue encroachment prevention member 676 contacts the vessel wall W, as the distal end 605 is moved across or within the vessel lumen VL, the tissue encroachment prevention member 676 maintains a separation of the aspiration lumen 606 and the wall tissue. This prevents tissue damage caused by vessel tissue being drawn into close proximity to a high pressure saline spray from the supply lumen. Additionally, the resiliency, flexibility, spring force, and elasticity of the tissue encroachment prevention member 676 provides a rebound force to move the distal end 605, and thereby the distal opening 607, away from the vessel wall W so as to avoid damaging the wall or vessel tissue. Inclusion of the coiled state of the tissue encroachment prevention member 676 allows passage of clot or aspirant A through the gaps 656 between adjacent coils of the coiled structure or through the channel 654 (FIG. 23) and into the aspiration lumen 606, thereby allowing aspiration to continue.

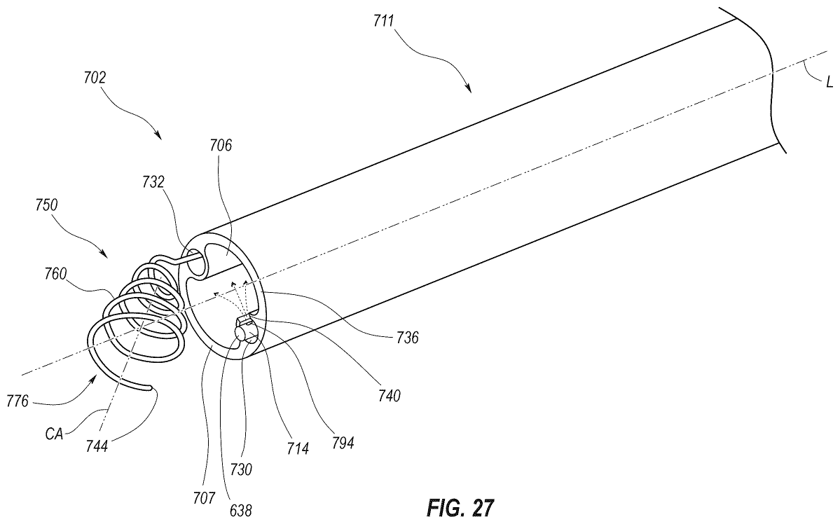

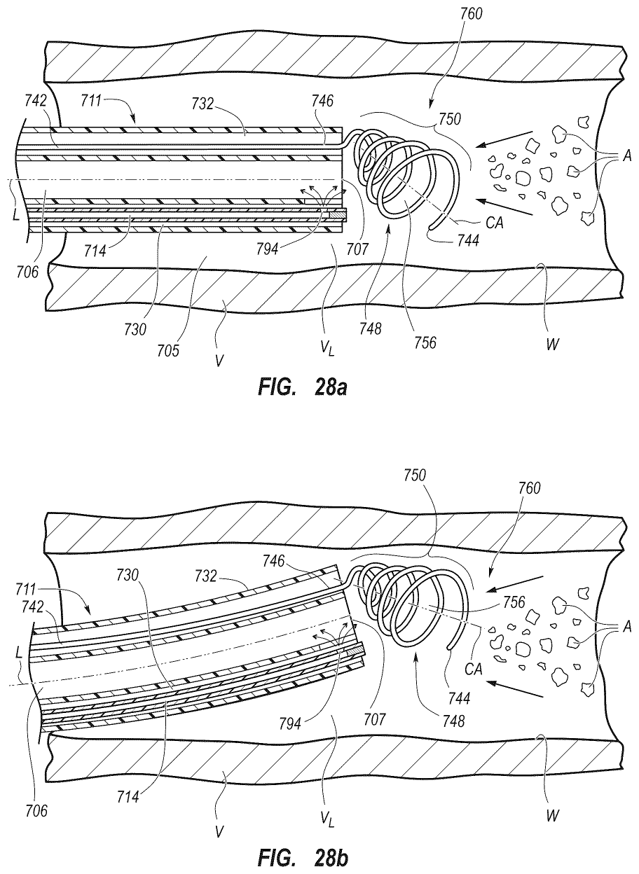

Turning to FIGS. 27-28b, illustrated is an aspiration catheter 702 having a tissue encroachment prevention assembly 750 that is deployable and retractable. Such tissue encroachment prevention assembly may provide a means for preventing the blood vessel from being drawn into the aspiration lumen and coming into close proximity to an orifice of a supply lumen (or orifices of one or more supply lumens) during aspiration. The aspiration catheter 702 can be any of the aspiration catheters described herein, such as aspiration catheters 102, 202, 302, 402, 502a, 502b, 502c, and 602 and so the disclosure related to aspiration catheters 102, 202, 302, 402, 502a, 502b, 502c, and 602 is also applicable to the aspiration catheter 702. Additionally, the disclosure related to the other tissue encroachment prevention assemblies is also applicable to the tissue encroachment prevention assembly 750.

As illustrated in FIG. 27, the aspiration catheter 702 includes a shaft 711 possessing three lumens, such as an aspiration lumen 706, a supply tube lumen 730 that receives a supply tube with the supply lumen 714, and a tissue encroachment prevention assembly lumen 732 that receives the tissue encroachment prevention assembly 750. The shaft 711 can be a multi-lumen extruded tube, such as a tube formed of a polymer, PEBAX, VESTAMID, nylon, polyurethane, combinations or modifications thereof, and optionally reinforced with braids, wires, etc. Alternatively, the shaft 711 can be a hypotube or other tubular member with tubular members disposed within the shaft 711 to form the supply tube lumen 730 and the tissue encroachment prevention assembly 750. Stated another way, the supply tube lumen 730 and the tissue encroachment prevention assembly 750 can be formed of tubular members that are disposed within the aspiration lumen 706 of the shaft 711.

The aspiration lumen 706 is formed towards a center of the shaft 711, in a similar manner to shaft 611, as illustrated in FIGS. 23 and 25b, with the supply tube lumen 730 and the tissue encroachment prevention assembly lumen 732 formed in a wall 736 of a shaft body 717a of the shaft 711.

With continued reference to FIGS. 27, disposed in the wall 736 towards a distal end 638 of the supply tube lumen 730 is an opening 740 that is aligned with an orifice 794 for ejection or jetting fluid into the aspiration lumen 706. The supply lumen 714 is connected to or fluidly coupled to a high pressure fluid source (e.g., SDU 212) to provide the fluid. This fluid traverses the aspiration lumen 706 to macerate or break up the thrombus, clot, aspirant, etc. that is drawn into the aspiration lumen 706.

Disposed within the tissue encroachment prevention assembly lumen 732 is the tissue encroachment prevention assembly 750. The tissue encroachment prevention assembly 750 can be advanced from within the tissue encroachment prevention assembly lumen 732 through movement of a handle or other actuator disposed at the handpiece 233 (FIG. 4). The tissue encroachment prevention assembly 750 includes an elongate member 742 with an atraumatic member 744, including a portion that transitions from a first state (similar to that discussed in relation to aspiration catheter 702) when retained within the tissue encroachment prevention assembly lumen 732 to a second state when the portion of the elongate member 742 is disposed from a distal end of the tissue encroachment prevention assembly lumen 732.

A first portion 746 of the elongate member 742 maintains the same shape or state inside or outside of the tissue encroachment prevention assembly lumen 732, while a second portion 748 of the elongate member 742, which includes a tissue encroachment prevention member 776, transitions to a second state in which the tissue encroachment prevention member 776 will prevent the vessel tissue from being drawn into close proximity to a high pressure saline spray from the supply lumen 714. As the tissue encroachment prevention member 776 transitions to the second state, the tissue encroachment prevention member 776 transitions to a coiled state having a coil axis CA that is transverse to a longitudinal axis L of the aspiration catheter 702 and forms an outwardly tapered coiled structure. While the tissue encroachment prevention member 776 extends across a distal opening 707 of the aspiration lumen 706, clot, thrombus or aspirant can still flow through gaps 756 between adjacent coils. The larger dimensioned or diameter coil distal end 760 provides a larger contact surface with vessel tissue to prevent the vessel tissue from being drawn towards the aspiration lumen 706 and into contact with the high pressure spray or jet from the orifice 794 of the supply lumen 714.

The elongate member 742 can be a wire, a hollow tube, a rod, combinations or modifications thereof that is formed to return to a pre-defined shape, i.e., the coiled state, following advancing the tissue encroachment prevention member 776 from the elongate member 742. For instance, the tissue encroachment prevention member 776, and the elongate member 742 as a whole, can be formed of a shape memory material, such as a shape member alloy or metal or a shape member polymer, a spring steel or alloy, combinations of modifications thereof. Alternatively, the tissue encroachment prevention member 776 can be formed of a material, or otherwise worked to form the pre-defined shape, while a reminder of the elongate member 742, such as first portion 746, is formed of a different material. For instance, the first portion 746 can be formed of stainless steel, while the tissue encroachment prevention member 776 is formed of Nitinol.

The coiled body of the tissue encroachment prevention member 776 has a pre-formed shape with one or more turns or coils, with the number and pitch of the coils being selected to allow clot, thrombus, or aspirant to pass through the gaps 756 and into the aspiration lumen 706. The pitch and number of the coils also can be varied to increase or decrease a flexibility of the tissue encroachment prevention member 776. The pitch can range from about 1 mm to about 2 mm, from about 2 mm to about 4 mm, from about 4 mm to about 6 mm, from about 6 mm to about 10 mm, from about 1 mm to about 10 mm, or is in a range between any two of the foregoing. Stated another way, the pitch can range from about 0.039″ to about 0.079″, from about 0.079″ to about 0.157″, from about 0.157″ to about 0.236″, from about 0.236″ to about 0.394″, from about 0.039″ to about 0.394″, or is in a range between any two of the foregoing. The number of turns or coils associated with the tissue encroachment prevention member 676 can range from about 2 to about 4 from about 4 to about 6, from about 6 to about 12, from about 2 to about 12, or is in a range between any two of the foregoing. The material selected for the tissue encroachment prevention member 776 can also aid with varying a resiliency, flexibility, spring force, and elasticity of the tissue encroachment prevention member 776 and its ability to rebound or move the distal end of the aspiration catheter 702 from a tissue wall.

Turning to FIG. 28a, the tissue encroachment prevention member 776 of the tissue encroachment prevention assembly 750 flexes to transition from the transverse orientation illustrated in FIG. 27 to a more proximally facing orientation, or bend proximally, when encountering clot or thrombus that is suctioned by the suction force created in the aspiration lumen 706. This allows the clot or thrombus to enter more deeply into the distal opening 707 of the aspiration lumen 706 and into a position to be macerated by the jet or stream from the orifice 794. While the tissue encroachment prevention member 776 can bend to allow the clot or thrombus to enter into the aspiration lumen 706, it has sufficient resiliency or elasticity to return to the transverse orientation.

Turning to FIG. 28b, the tissue encroachment prevention assembly 750 is illustrated preventing tissue being drawn into the aspiration lumen 706. As the aspiration catheter 702 is moved to a position to aspirate clot, thrombus, or aspirant A, a clinician can advance the tissue encroachment prevention member 776 from the tissue encroachment prevention assembly lumen 732 and from the distal opening 734. As the tissue encroachment prevention member 776 exits the distal opening 734, it returns to the pre-set or pre-formed shape. When the tissue encroachment prevention member 776 contacts the vessel wall W when the distal end 705 is moved across or within the vessel lumen VL, the tissue encroachment prevention member 776 maintains a separation of the aspiration lumen 706 and the wall tissue. This prevents tissue damage caused by vessel tissue being drawn into close proximity to high pressure saline spray from the supply lumen 714. Additionally, the resiliency, flexibility, spring force, and elasticity of the tissue encroachment prevention member 776 provides a rebound force to move the distal end 705, and thereby the distal opening 707, away from the vessel wall W so as to avoid damaging the wall or vessel tissue.

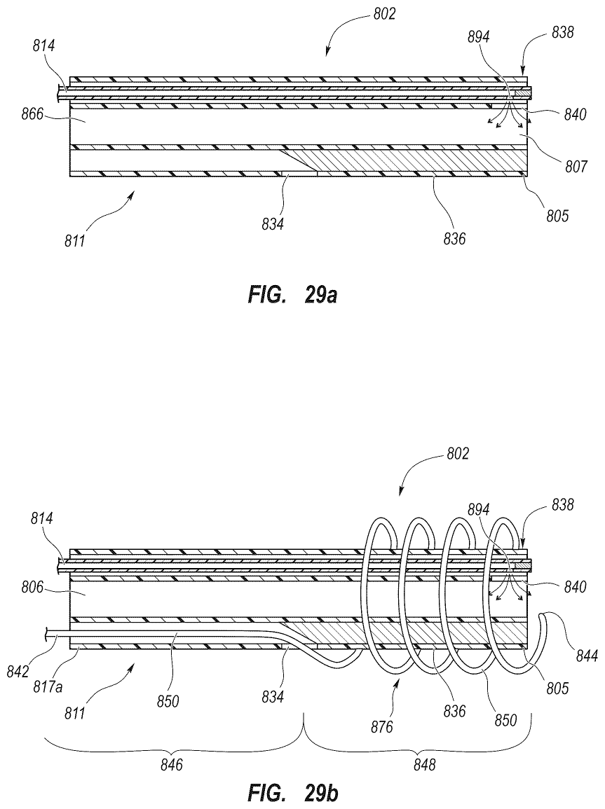

Turning to FIGS. 29a-31b, illustrated is an aspiration catheter 802 having a deployable and retractable tissue encroachment prevention assembly 850. Such tissue encroachment prevention assembly may provide a means for preventing the blood vessel from being drawn into the aspiration lumen and coming into close proximity to an orifice of a supply lumen (or orifices of one or more supply lumens) during aspiration. The aspiration catheter 802 can be any of the aspiration catheters described herein, such as aspiration catheters 102, 202, 302, 402, 502a, 502b, 502c, 602, and 702 and so the disclosure related to aspiration catheters 102, 202, 302, 402, 502a, 502b, 502c, 602, and 702 is also applicable to the aspiration catheter 802. Additionally, the disclosure related to the other tissue encroachment prevention assemblies is also applicable to the tissue encroachment prevention assembly 850.

As illustrated in FIG. 29a, the aspiration catheter 802 includes a shaft 811 possessing three lumens, such as an aspiration lumen, a supply tube lumen 830 that receives a supply tube with the supply lumen 814, and a tissue encroachment prevention assembly lumen 832 that receives the tissue encroachment prevention assembly 850. The shaft 811 can be a multi-lumen extruded tube, such as a tube formed of a polymer, PEBAX, VESTAMID, nylon, polyurethane, combinations or modifications thereof, and optionally reinforced with braids, wires, etc. Alternatively, the shaft 811 can be a hypotube or other tubular member with tubular members disposed within the shaft 811 to form the supply tube lumen 830 and the tissue encroachment prevention assembly 850. Stated another way, the supply tube lumen 830 and the tissue encroachment prevention assembly 850 can be formed of tubular members that are disposed within the aspiration lumen 806 of the shaft 811.

Unlike the aspiration catheters 602 and 702, however, the tissue encroachment prevention assembly lumen 832 terminates proximal a distal opening 807 of a distal end 805 of the aspiration catheter 802. Instead, the tissue encroachment prevention assembly lumen 832 extends through a wall 836 of the shaft 811, with a tissue encroachment prevention assembly lumen distal opening 834 facing outwardly from the wall 836—i.e., the tissue encroachment prevention assembly lumen distal opening 834 faces in a direction transverse to a direction in which the distal opening 807 of the aspiration lumen 806 faces. This allows a tissue encroachment prevention member 876 to exit the aspiration catheter 802 proximal the distal end 805 and/or the distal opening 807, as will be described in more detail hereinafter.

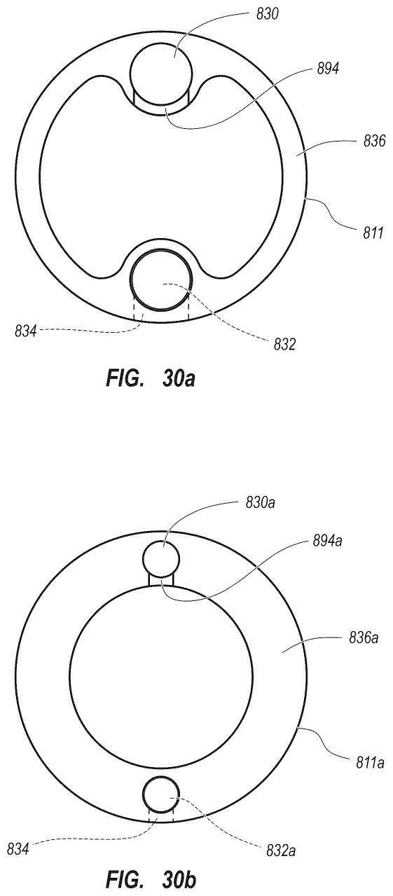

The aspiration lumen 806 is formed towards a center of the shaft 811, such as illustrated in FIGS. 29a and 30b, with the supply tube lumen 830 and the tissue encroachment prevention assembly lumen 832 formed in a wall 836 of a shaft body 817a of the shaft 811. As illustrated in FIG. 30a, for instance, the wall 836 protrudes inwardly towards a center of the aspiration lumen 806 to accommodate the supply tube lumen 830 and the tissue encroachment prevention assembly lumen 832. However, such as illustrated in FIG. 30b, in other configurations, the wall 836a does not protrude inwardly towards the center to accommodate the supply tube lumen 830 and the tissue encroachment prevention assembly lumen 832.

With continued reference to FIGS. 29a-30b, disposed in the wall 836 towards a distal end 838 of the supply tube lumen 830 is an opening 840 that is aligned with an orifice 894 for ejection or jetting fluid into the aspiration lumen 806. The supply lumen 814 is connected to or fluidly coupled to a high pressure fluid source (e.g., SDU 212) to provide the fluid. This fluid traverses the aspiration lumen 806 to macerate or break up the thrombus, clot, aspirant, etc. that is drawn into the aspiration lumen 806.

Disposed within the tissue encroachment prevention assembly lumen 832 is the tissue encroachment prevention assembly 850. The tissue encroachment prevention assembly 850 can be advanced from within the tissue encroachment prevention assembly lumen 832 through movement of a handle or other actuator disposed at the handpiece 233 (FIG. 4). The tissue encroachment prevention assembly 850 includes an elongate member 842 with an atraumatic member 844, including a portion that transitions from a first state when retained within the tissue encroachment prevention assembly lumen 832 (see FIG. 29a) to a second state when the portion of the elongate member 842 is disposed from a distal end of the tissue encroachment prevention assembly lumen 832 (see FIG. 29b). A first portion 846 of the elongate member 842 maintains the same shape or state inside or outside of the tissue encroachment prevention assembly lumen 832, while a second portion 848 of the elongate member 842, which includes a tissue encroachment prevention member 876, transitions to a second state in which the tissue encroachment prevention member 876 wraps around the aspiration catheter 802 and extends to, and optionally past, the distal end 805 of the aspiration catheter 802 to prevent the vessel tissue from being drawn into close proximity to the high pressure saline spray from the supply lumen 814. In other configurations, however, the coil axis CA of the coiled state of the tissue encroachment prevention member 876 are not coincident with the longitudinal axis L of the aspiration lumen 806, but are offset from, and optionally parallel or transverse to, the longitudinal axis L. For instance, in the coiled state the tissue encroachment prevention member 876 may be offset to one side so that the tissue encroachment prevention member 876 is preferentially positioned towards the supply tube lumen side of the aspiration catheter 802 or towards the tissue encroachment prevention tube lumen side of the aspiration catheter 802.

The elongate member 842 can be a wire, a hollow tube, a rod, combinations or modifications thereof that is formed to return to a pre-defined shape, i.e., the coiled state, following advancement of the tissue encroachment prevention member 876 from the elongate member 842. For instance, the tissue encroachment prevention member 876, and the elongate member 842 as a whole, can be formed of a shape memory material, such as a shape member alloy or metal or a shape member polymer, a spring steel or alloy, combinations of modifications thereof.

Alternatively, the tissue encroachment prevention member 876 can be formed of a material, or otherwise worked to form the pre-defined shape, while a reminder of the elongate member 842, such as first portion 846, is formed of a different material. For instance, the first portion 846 can be formed of stainless steel, while the tissue encroachment prevention member 876 is formed of Nitinol.

A pitch and number of the coils also can be varied to increase or decrease a flexibility of the tissue encroachment prevention member 876. The pitch can range from about 1 mm to about 2 mm, from about 2 mm to about 4 mm, from about 4 mm to about 6 mm, from about 6 mm to about 10 mm, from about 1 mm to about 10 mm, or is in a range between any two of the foregoing. Stated another way, the pitch can range from about 0.039″ to about 0.079″, from about 0.079″ to about 0.157″, from about 0.157″ to about 0.236″, from about 0.236″ to about 0.394″, from about 0.039″ to about 0.394″, or is in a range between any two of the foregoing. The number of turns or coils associated with the tissue encroachment prevention member 876 can range from about 2 to about 4 from about 4 to about 6, from about 6 to about 12, from about 2 to about 12, or is in a range between any two of the foregoing. The material selected for the tissue encroachment prevention member 876 can also aid with varying a resiliency, flexibility, spring force, and elasticity of the tissue encroachment prevention member 876 and its ability to rebound or move the distal end of the aspiration catheter 802 from a tissue wall.

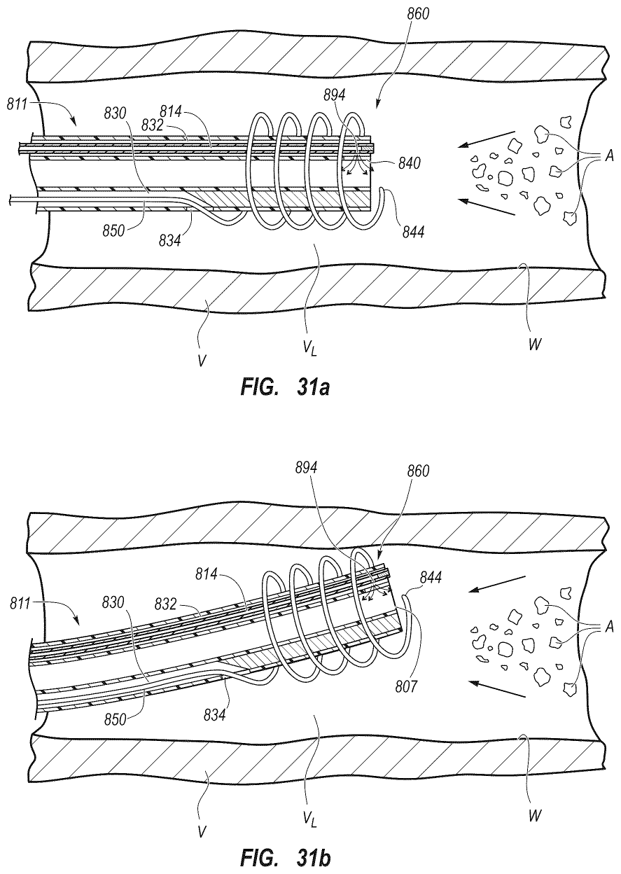

Turning to FIGS. 31a and 31b, the tissue encroachment prevention assembly 850 is illustrated preventing tissue being drawn into the aspiration lumen 806. As the aspiration catheter 802 is moved to a position to aspirate a clot, thrombus, or aspirant A, a clinician can advance the tissue encroachment prevention member 876 from the tissue encroachment prevention assembly lumen 832 and from the distal opening 834. As the tissue encroachment prevention member 876 exits the distal opening 834, it returns to the pre-set or pre-formed shape and coils or wraps around the aspiration catheter 802. When the tissue encroachment prevention member 876 contacts the vessel wall W, as the distal end 805 is moved across or within the vessel lumen VL, the tissue encroachment prevention member 876 maintains a separation of the aspiration lumen 806 and the wall tissue. This prevents tissue damage caused by vessel tissue being drawn into close proximity to the high pressure saline spray from the supply lumen. Additionally, the resiliency, flexibility, spring force, and elasticity of the tissue encroachment prevention member 876 provides a rebound force to move the distal end 805, and thereby the distal opening 807, away from the vessel wall W so as to avoid damaging the wall or vessel tissue.

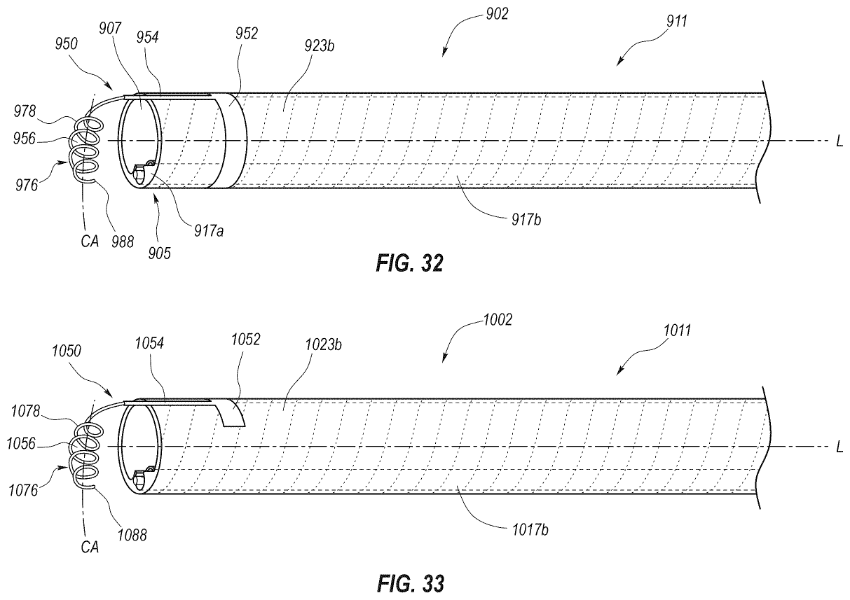

Turning to FIG. 32, illustrated is an aspiration catheter 902 having a tissue encroachment prevention assembly 950. Such tissue encroachment prevention assembly may provide a means for preventing the blood vessel from being drawn into the aspiration lumen and coming into close proximity to an orifice of a supply lumen (or orifices of one or more supply lumens) during aspiration. The aspiration catheter 902 can be any of the aspiration catheters described herein, such as aspiration catheters 102, 202, 302, 402, 502a, 502b, 502c, 602, 702, and 802 and so the disclosure related to aspiration catheters 102, 202, 302, 402, 502a, 502b, 502c, 602, 702, and 802 is also applicable to the aspiration catheter 902. Additionally, the disclosure related to the other tissue encroachment prevention assemblies is also applicable to the tissue encroachment prevention assembly 950.

The aspiration catheter 902 provides a barrier to tissue being drawn into an aspiration lumen, thereby controlling or limiting access to the aspiration lumen of the aspiration catheter such that tissue does not encroach or be drawn into the aspiration lumen and towards the high pressure spray from the supply lumen, while thrombus or aspirant is still capable of being collected.

As illustrated, the aspiration catheter 902 includes a shaft 911 with a tissue encroachment prevention assembly 950 disposed towards a distal end 905 of the shaft 911, with a portion of the tissue encroachment prevention assembly 950 extending distal a distal opening 907 of the aspiration catheter 902. A jacket 917b extends along the shaft 911, with the tissue encroachment prevention assembly 950 mounted to the jacket 917b. While illustrated as being mounted to the jacket 917b, it is understood that the tissue encroachment prevention assembly 950 can be mounted to a shaft body 917a of the shaft 911, optionally with the jacket 917b overlapping a portion of the tissue encroachment prevention assembly 950.

As illustrated, the tissue encroachment prevention assembly 950 extends from the shaft 911 and includes a base 952, an elongate member 954 extending from the base 952, and a tissue encroachment prevention member 976. The base 952 extends circumferentially around the shaft 911 and has a profile approximating a curvature of the outer surface 923b of the shaft 911.

In an alternative embodiment illustrated in FIG. 33, the base 1052 extends partially around the outer surface 1023b—i.e., it forms a portion of a cylinder or ring (the elongate member 1054 and the tissue encroachment prevention member 1076 can be similar to the elongate member 954 and the tissue encroachment prevention member 976, respectively, of FIG. 32). In the alternate configuration of FIG. 33, in contrast to the tissue encroachment prevention assemblies in FIGS. 9a-18 where the base is disposed between two jackets or in some other position, the base 1052 is mounted to the jacket 1017b of a shaft 1011 of the aspiration catheter 1002. For instance, the base 1052 can mount to jacket 1017b through an adhesive, thermal bond, mechanical fastener attachment, combinations or modifications thereof. Alternatively, when the base 1052 is formed of a material complementary to the material forming the jacket 1017b, the base 1052 and the jacket 1017b can be thermally bonded where both melt to form a monolithic, unitary structure or junction. Such tissue encroachment prevention assembly may provide a means for preventing the blood vessel from being drawn into the aspiration lumen and coming into close proximity to an orifice of a supply lumen during aspiration.

Returning to FIG. 32, the elongate member 954 extends from the base 952 and beyond the distal end 905 of the aspiration catheter 902. An end 978 of the elongate member 954 extends towards the longitudinal axis L to aid positioning the tissue encroachment prevention member 976 in line within the distal opening 907. This elongate member 954 can either be mounted to the jacket 917b, or other portion of the shaft 911, or can be free to move with movement of the tissue encroachment prevention member 976 as the tissue encroachment prevention member 976 engages with clot, thrombus, or aspirants or vessel tissue. In the latter case, the elongate member 954 and the tissue encroachment prevention member 976 are connected to the base 952 in a cantilever fashion.

Disposed at an end 978 of the elongate member 954 is the tissue encroachment prevention member 976 having a coiled body 988 with a coil axis CA that is transverse to a longitudinal axis L of the aspiration catheter 902. The coiled body 988 has a pre-formed shape with one or more turns or coils, with the number and pitch of the coils being selected to allow clot, thrombus, or aspirant to pass through the gaps 956 and into the aspiration lumen 906. The pitch and number of the coils also can be varied to increase or decrease a flexibility of the tissue encroachment prevention member 976. The pitch can range from about 1 mm to about 2 mm, from about 2 mm to about 4 mm, from about 4 mm to about 6 mm, from about 6 mm to about 10 mm, from about 1 mm to about 10 mm, or is in a range between any two of the foregoing. Stated another way, the pitch can range from about 0.039″ to about 0.079″, from about 0.079″ to about 0.157″, from about 0.157″ to about 0.236″, from about 0.236″ to about 0.394″, from about 0.039″ to about 0.394″, or is in a range between any two of the foregoing. The number of turns or coils associated with the tissue encroachment prevention member 976 can range from about 2 to about 4 from about 4 to about 6, from about 6 to about 12, from about 2 to about 12, or is in a range between any two of the foregoing. The material selected for the tissue encroachment prevention member 976 can also aid with varying a resiliency, flexibility, spring force, and elasticity of the tissue encroachment prevention member 976 and its ability to rebound or move the distal end of the aspiration catheter 902 from a tissue wall. The base 952, the elongate member 954, and the tissue encroachment prevention member 976 can be of a metal, alloy, shape-memory material, such as a shape member alloy or metal or a shape member polymer, a composite, a polymer, or combinations or modifications thereof. Additionally, the elongate member 954 and the tissue encroachment prevention member 976 can be a wire, a hollow tube, a rod, combinations or modifications thereof that is formed to return to a pre-defined shape, i.e., the coiled state.

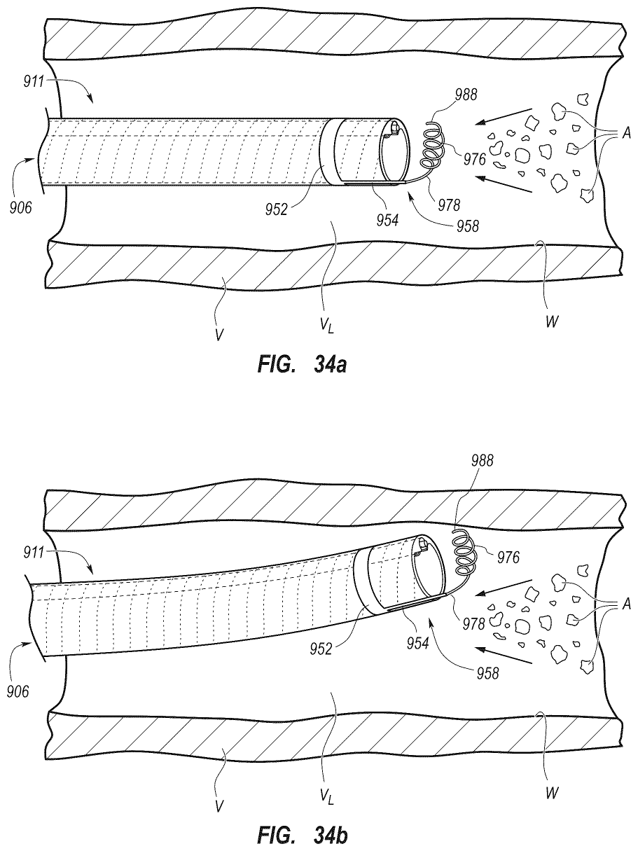

Turning to FIGS. 34a and 34b, the tissue encroachment prevention assembly 950 is illustrated preventing tissue being drawn into the aspiration lumen 906. As the aspiration catheter 902 is moved to a position to aspirate a clot, thrombus, or aspirant A by a clinician, the tissue encroachment prevention member 976, having the pre-set or pre-formed shape, extends distally from the distal end 905 of the aspiration catheter 902. When the tissue encroachment prevention member 976 contacts the vessel wall W when the distal end 905 is moved across or within the vessel lumen VL, the tissue encroachment prevention member 976 maintains a separation of the aspiration lumen 906 and the wall tissue. This prevents tissue damage caused by vessel tissue being drawn into close proximity to the high pressure saline spray from the supply lumen. Additionally, the resiliency, flexibility, spring force, and elasticity of the tissue encroachment prevention member 976 provides a rebound force to move the distal end 905, and thereby the distal opening 907, away from vessel wall W so as to avoid damaging the wall or vessel tissue. Inclusion of the coiled state of the tissue encroachment prevention member 976 allows passage of clot or aspirant A through the gaps 956 between adjacent coils of the coiled structure and into the aspiration lumen 906, thereby allowing aspiration to continue.

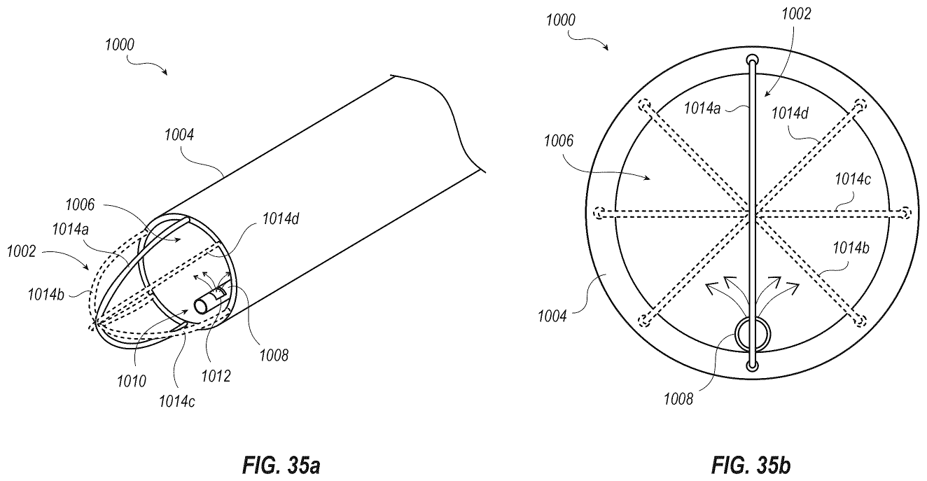

Attention is now directed to FIGS. 35a-37c, which illustrate an aspiration catheter 1000 having a tissue encroachment prevention assembly 1002. In some embodiments, the tissue encroachment prevention assembly 1002 may be in a fixed or permanently deployed configuration. In other embodiments, the tissue encroachment prevention assembly 1002 may be selectively deployable and retractable. In either case, the tissue encroachment prevention assembly 1002 may provide a means for preventing the blood vessel from being drawn into the aspiration lumen and coming into close proximity to an orifice of a supply lumen during aspiration.

The aspiration catheter 1000 can be any of the aspiration catheters described herein, so the disclosure related to the other aspiration catheters disclosed herein is also applicable to the aspiration catheter 1000. Additionally, the disclosure related to the other tissue encroachment prevention assemblies is also applicable to the tissue encroachment prevention assembly 1002.

As illustrated in FIGS. 35a and 35b, the aspiration catheter 1000 includes a shaft 1004. The shaft 1004 includes an aspiration lumen 1006. Disposed within the aspiration lumen 1006 is a supply tube 1008 having a supply lumen 1010. Disposed in a wall of the supply tube 1008 towards a distal end thereof is an orifice 1012 for ejection or jetting fluid into the aspiration lumen 1006. The supply lumen 1010 is connected to or fluidly coupled to a high pressure fluid source (e.g., SDU 212) to provide the fluid. This fluid traverses the aspiration lumen 1006 to macerate or break up the thrombus, clot, aspirant, etc. that is drawn into the aspiration lumen 1006.

While the supply tube 1008 is illustrated as being positioned within the aspiration lumen 1006, this is only exemplary. As with other embodiments, disclose herein, the supply tube 1008 may be disposed within a lumen in the wall of the shaft 1004. In either case, the shaft 1004 can be a single-or multi-lumen extruded tube, such as a tube formed of a polymer, PEBAX, VESTAMID, nylon, polyurethane, combinations or modifications thereof, and optionally reinforced with braids, wires, etc. Alternatively, the shaft 1004 can be a hypotube or another tubular member.

The tissue encroachment prevention assembly 1002 includes one or more atraumatic members. FIGS. 35a and 35b illustrate an atraumatic member 1014a and optional atraumatic members 1014b, 1014c, 1014d (shown in dashed lines). Each of the atraumatic members extends across the distal opening to the aspiration lumen 1006. In the illustrated embodiment, the atraumatic members span the opening to the aspiration lumen 1006. In some embodiments, one or more of the atraumatic members extend between opposing sides of the shaft 1004 that are 180° apart, are arranged parallel to a diameter of the aspiration lumen 1006, and/or extend through a central axis of the aspiration lumen 1006. However, in other embodiments, one or more of the atraumatic members may be arranged differently. For instance, one or more of the atraumatic members may extend between portions of the shaft 1004 that are less or greater than 180° apart, are arranged non-parallel to a diameter of the aspiration lumen 1006, and/or do not extend through a central axis of the spiration lumen 1006. Likewise, while FIGS. 35a and 35b illustrate the atraumatic members crossing or intersecting one another at or near the central axis of the aspiration lumen 1006, such is not required. Rather, in other embodiments, the atraumatic members may cross or intersect one another at a location that is offset from the central axis of the aspiration lumen 1006. Still further, one or more of the atraumatic members may not cross or intersect other atraumatic members. In some embodiments, multiple atraumatic members are included and at least some of the atraumatic members are arranged parallel to one another.

In some embodiments, the orientation of at least one of the atraumatic members may be determined at least in part by the direction of the spray from the orifice 1012 of the supply tube 1008. For instance, as shown in FIGS. 35a and 35b, the atraumatic member 1014a may be oriented generally parallel to the general direction of the spray from the orifice 1012. This is merely exemplary. For instance, the atraumatic members 1014c is oriented generally perpendicular to the direction of the spray. Still further, the atraumatic members 1014b and 1014d are oriented neither parallel nor perpendicular to the direction of the spray.

As shown in FIGS. 35a and 35b, the atraumatic members may have an arcuate shape when viewed from the side. In some embodiments, the proportions of the atraumatic members may be such that the length thereof (e.g., the distance between the distal end of the shaft 1004 and the distal most portion of the atraumatic members (e.g., at the vertex of the arcuate shape)) is greater than the width thereof (e.g., the distance between the opposing ends of the atraumatic member that are connected to or otherwise associated with the shaft 1004). In such case, the atraumatic member may have a pointed, albeit still rounded, tip. In other embodiments, the length of the atraumatic member may be less than the width, providing the atraumatic member flatter end profile. In still other embodiments, the length and the width of the atraumatic member may be equal, providing the atraumatic member with a semi-circular profile.

As alluded to above, the atraumatic members may be connected to the shaft 1004 in a fixed configuration. For instance, opposing ends of the atraumatic members may be secured to an interior surface of the aspiration lumen 1006, an exterior wall of the shaft 1004, or to the distal end of the shaft 1004. The attachment between the atraumatic members and the shaft 1004 may made via any suitable mean, including adhesives, welding, or the like. In some embodiments, the shaft 1004 may include recesses in the distal end thereof. The ends of the atraumatic members may be inserted and retained within the recesses.

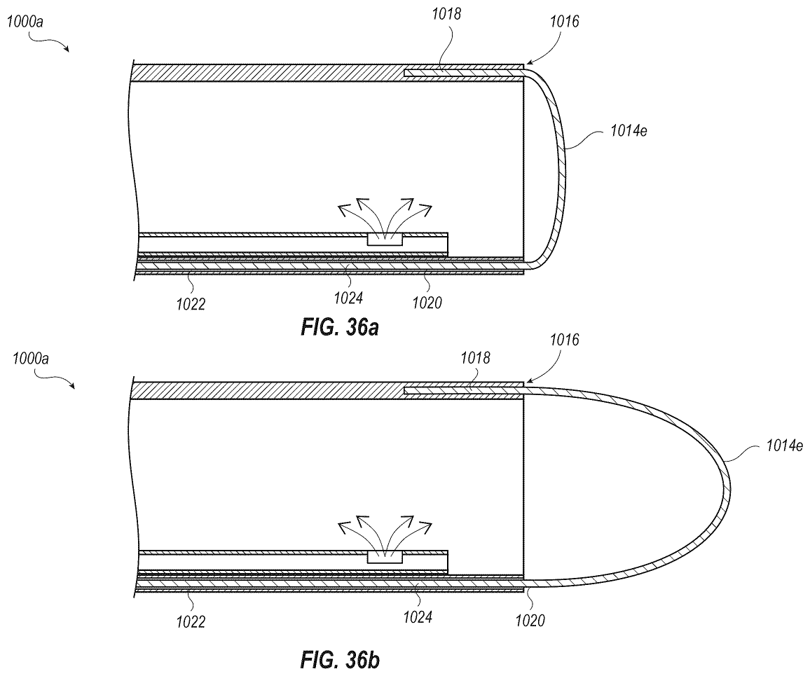

In other embodiments, the atraumatic members may be movably associated with the shaft of the aspiration catheter such that they can be selectively moved between retracted and deployed states or configurations. FIGS. 36a and 36b illustrate on example embodiment showing how the atraumatic members may be selectively moved between retracted and deployed states. The embodiment of FIGS. 36a and 36b may be similar or identical to that of FIGS. 35a and 35b in many respects. Accordingly, the following discussion of FIGS. 36a and 36b will focus on those aspects that may be different.

As shown in FIGS. 36a and 36b, the aspiration catheter 1000a includes a recess 1016 that extends into the distal end thereof. A first end 1018 of the atraumatic member 1014e may be received within the recess 1016. In some embodiments, the first end 1018 is permanently secured within the recess 1016, such that the first end 1018 remains in the recess 1016 when the atraumatic member 1014e is in both the retracted state and the deployed. In other embodiments, the first end 1018 may be selectively inserted into the recess 1016 when the atraumatic member 1014e is moved to the deployed state and removed from the recess 1016 when the atraumatic member 1014e is moved to the retracted state.

An opposing second end 1020 of the atraumatic member 1014e may extend into a tissue encroachment prevention assembly lumen 1022 in the shaft 1004a. Extending proximally from the second end 1020 through the tissue encroachment prevention assembly lumen 1022 is an elongate member 1024.

The atraumatic member 1014e may be moved between the retracted state (FIG. 36a) and the deployed state (FIG. 36b) through movement of a handle or other actuator disposed at the handpiece 233 (FIG. 4). When the atraumatic member 1014e is in the retracted state, the second end 1020 and at least a portion of the atraumatic member 1014e between the first and second ends 1018, 1020 is disposed within the tissue encroachment prevention assembly lumen 1022, as shown in FIG. 36a. Movement of the handle or other actuator at the handpiece 233 causes the elongate member 1024 to move distally through the tissue encroachment prevention assembly lumen 1022, which advances the atraumatic member 1014e out of the tissue encroachment prevention assembly lumen 1022.

As the atraumatic member 1014e advances out of the tissue encroachment prevention assembly lumen 1022, the shape of the atraumatic member 1014e (or a portion thereof) may transition from a first state or shape (generally straight) when retained within the tissue encroachment prevention assembly lumen 1022 (see FIG. 36a) to a second state or shape (arcuate) when disposed outside of the tissue encroachment prevention assembly lumen 1022 (see FIG. 36b). When the atraumatic member 1014e has transitioned to the second state or shape, the atraumatic member 1014e may prevent the vessel tissue from being drawn into close proximity to a high pressure saline spray from the supply lumen 1026.

The elongate member 1024 can be a wire, a hollow tube, a rod, combinations or modifications thereof. Similarly, the atraumatic member 1014e may be formed of a wire, a hollow tube, a rod, combinations or modifications thereof that is configured to return to a pre-defined shape, i.e., the arcuate shape, following advancing the atraumatic member 1014e from the tissue encroachment prevention assembly lumen 1022. In some embodiments, the atraumatic member 1014e may be formed of Nitinol. The material selected for the atraumatic member 1014e can also aid with varying a resiliency, flexibility, spring force, and elasticity of the atraumatic member 1014e and its ability to rebound or move the distal end of the aspiration catheter 1000a from a tissue wall.

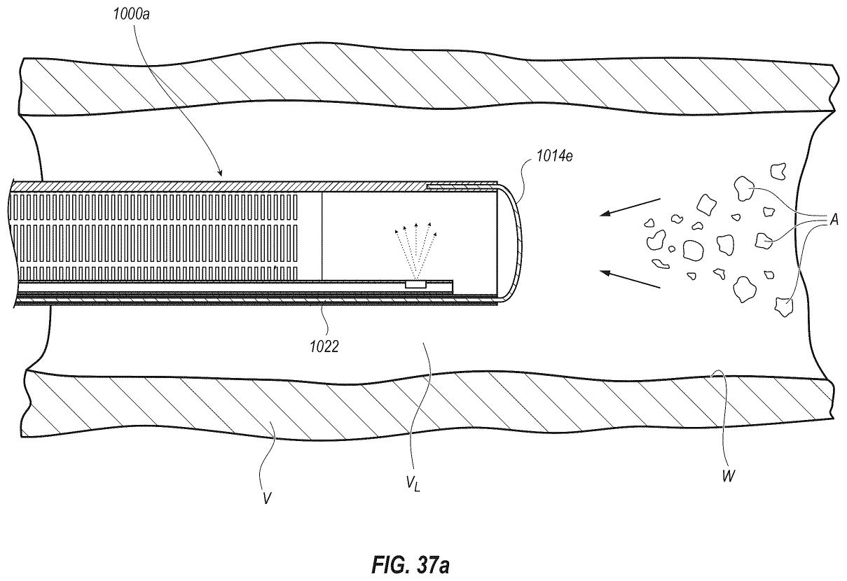

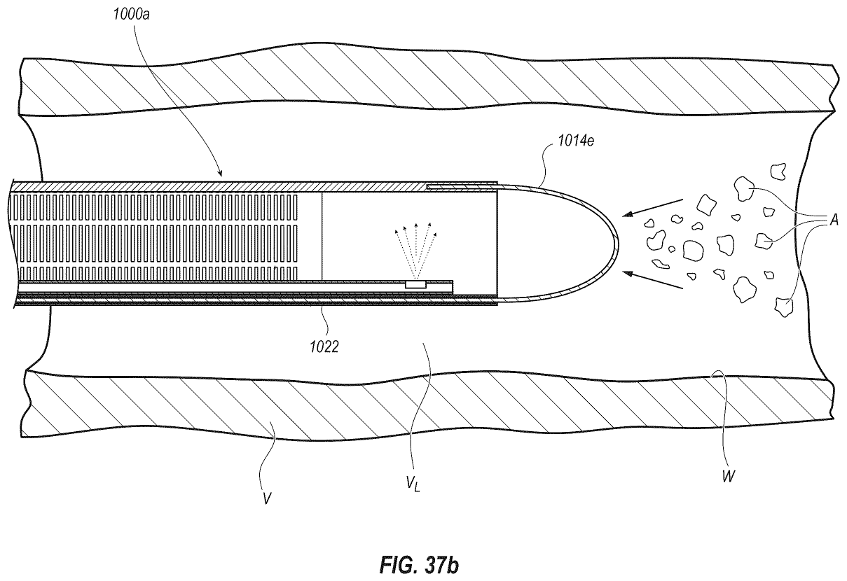

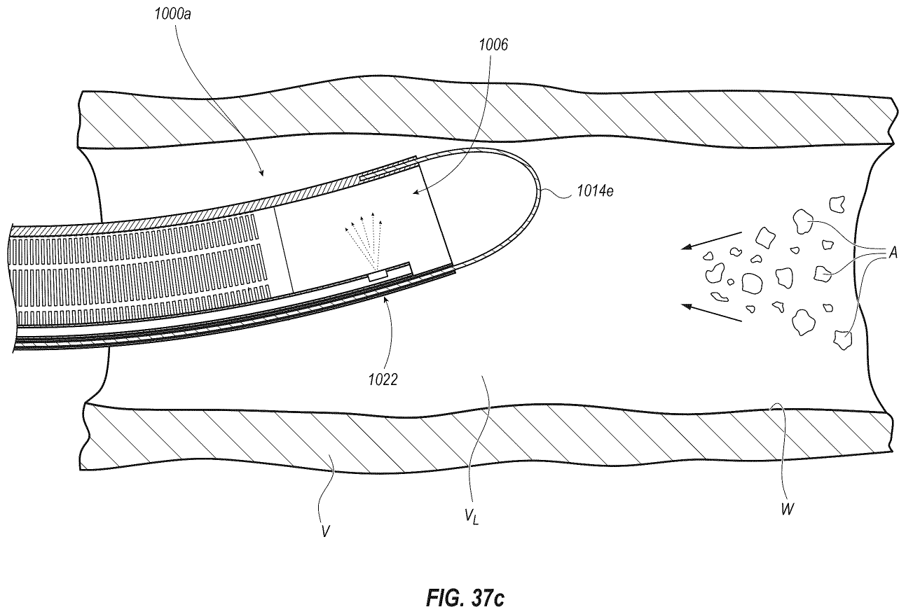

Turning to FIGS. 37a, 37b, and 37c, the aspiration catheter 1000a is illustrated within a vessel to aspirate a clot, thrombus, or aspirant A. As shown in FIG. 37a, the aspiration catheter 1000a may be positioned within a vessel lumen VL while the atraumatic member 1014e is in the retracted state. As the aspiration catheter 1000a is moved to a position to aspirate the clot, thrombus, or aspirant A, a clinician can advance the atraumatic member 1014e from the tissue encroachment prevention assembly lumen 1022 to the deployed state shown in FIG. 37b. As the atraumatic member 1014e is moved to the deployed state, it returns to the pre-set or pre-formed arcuate shape.

When the atraumatic member 1014e contacts the vessel wall W, as the aspiration catheter 1000a is moved within the vessel lumen VL, the atraumatic member 1014e maintains a separation of the aspiration lumen 1006 and the wall tissue. This prevents tissue damage caused by vessel tissue being drawn into close proximity to a high pressure saline spray from the supply lumen. Additionally, the resiliency, flexibility, spring force, and elasticity of the atraumatic member 1014e provides a rebound force to move the distal end of the aspiration catheter 1000a, and thereby the distal opening of the aspiration lumen 1006, away from the vessel wall W so as to avoid damaging the wall or vessel tissue.

The gaps or spaces around the atraumatic member 1014e and/or between the atraumatic member 1014e and other atraumatic members allows passage of clot or aspirant A through the gaps or spaces and into the aspiration lumen, thereby allowing aspiration to continue.

Although the atraumatic members are shown as having a generally smooth arcuate shape, this is only exemplary. In other embodiments, while the atraumatic members may have a generally arcuate shape, the atraumatic members may include waves, spirals, or other shapes or contours along the arcuate shape.

In some embodiments, the atraumatic members may be formed of a radiopaque material, such as a material or alloy containing a material with a higher atomic mass such as tantalum, tungsten, platinum/iridium, gold, silver, and so forth

Although the systems for aspirating thrombus described herein are predominantly focused on aspiration, the systems may also, or alternatively, be configured for injecting or infusing fluids, with or without drugs, and may incorporate related features described in U.S. Pat. No. 10,716,583, issued Jul. 21, 2020, and entitled, “Systems and Methods for Removal of Blood and Thrombotic Material” and U.S. Pat. No. 10,492,805, issued Dec. 3, 2019, and entitled, “Systems and Methods for Thrombosis and Delivery of an Agent.”

It is contemplated that various combinations or sub-combinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

Following are some further example embodiments of the invention. These are presented only by way of example and are not intended to limit the scope of the invention in any way. Further, any example embodiment can be combined with one or more of the example embodiments.

Embodiment 1. An aspiration catheter comprising an elongate shaft configured for placement within a blood vessel, a supply lumen and an aspiration lumen each extending along the shaft, the supply lumen having a proximal end and a distal end, and the aspiration lumen having a proximal end and a distal opening, an orifice at or near the distal end of the supply lumen, the orifice being configured to allow injection of pressurized fluid into the aspiration lumen at or near the distal end of the aspiration lumen when the pressurized fluid is delivered through the supply lumen; and a tissue encroachment prevention assembly for preventing the blood vessel from being drawn into said aspiration lumen and coming into close proximity to the orifice of the supply lumen during aspiration.

Embodiment 2. The aspiration catheter of embodiment 1, further comprising one or more supply lumens, each having an orifice configured to allow injection of pressurized fluid into the aspiration lumen at or near the distal end of the aspiration lumen when the pressurized fluid is pumped through the supply lumen.

Embodiment 3. The aspiration catheter of any of embodiments 1-2, wherein the elongate shaft is a laser cut hypotube laminated by a polymer.

Embodiment 4. The aspiration catheter of any of embodiments 1-3, wherein the elongate shaft is a multi-lumen extruded polymer shaft.

Embodiment 5. The aspiration catheter of any of embodiments 1-4, further comprising a radiopaque ring near the distal end of the aspiration catheter.

Embodiment 6. The aspiration catheter of any of embodiments 1-5, wherein said tissue encroachment prevention assembly comprises a plurality of tines extending from a point at or near the opening at the distal end of the elongate shaft, the plurality of tines each having distal ends that are attached to a spherical ball and proximal ends that extend from the distal end of the elongate shaft, wherein the plurality of tines attached to the spherical ball are configured to form a cage-like structure that prevents vessel tissue from being drawn into the aspiration lumen and into close proximity to the orifice of the supply lumen.

Embodiment 7. The aspiration catheter of any of embodiments 1-6, wherein at least 3 tines extend from the aspiration lumen.

Embodiment 8. The aspiration catheter of any of embodiments 1-7, wherein the tines contain a spring element that allows the tines to bend.

Embodiment 9. The aspiration catheter of any of embodiments 1-8, further comprising a radiopaque ring encircling the catheter near the distal end of the aspiration catheter.

Embodiment 10. The aspiration catheter of any of embodiments 1-9, wherein the ring has holes dispersed throughout.

Embodiment 11. The aspiration catheter of any of embodiments 1-10, wherein a width of the tines is tapered towards the spherical ball.

Embodiment 12. The aspiration catheter of any of embodiments 1-11, wherein the tines are laser welded or soldered directly to a surface of the spherical ball.

Embodiment 13. The aspiration catheter of any of embodiments 1-12, wherein the spherical ball comprises a blind hole to receive the tines.

Embodiment 14. The aspiration catheter of any of embodiments 1-3, wherein the spherical ball is made of a radiopaque material.

Embodiment 15. The aspiration catheter of any of embodiments 1-14, further comprising a mesh configuration at the proximal ends of the tines.

Embodiment 16. An aspiration catheter comprising an elongate shaft configured for placement within a blood vessel, the elongate shaft having a distal end with an opening at the distal end, a supply lumen and an aspiration lumen each extending along the shaft, the supply lumen having a proximal end and a distal end, and the aspiration lumen having a proximal end and a distal opening, an orifice near the distal end of the supply lumen, the orifice being configured to allow injection of pressurized fluid into the aspiration lumen at or near the distal end of the aspiration lumen when the pressurized fluid is delivered through the supply lumen, and a means for preventing the blood vessel from being drawn into the aspiration lumen and into close proximity to the orifice of the supply lumen during aspiration.

Embodiment 17. The aspiration catheter of embodiment 16, wherein the means for preventing the blood vessel from being drawn into the aspiration lumen comprises a cage-like structure extending distally from a point at or near the opening at the distal end of the elongate shaft.

Embodiment 18. The aspiration catheter of any of embodiments 16-17, wherein the cage-like structure comprises a plurality of tines, the plurality of tines each having distal ends that are attached to a spherical ball to form the cage-like structure and proximal ends that extend from the distal end of the elongate shaft.

Embodiment 19. An aspiration catheter comprising an elongate shaft configured for placement within a blood vessel, the elongate shaft having a distal end with an opening at the distal end, a supply lumen and an aspiration lumen each extending along an interior of the shaft, coextensive with the shaft, the supply lumen having a proximal end and a distal end, and the aspiration lumen having a proximal end and a distal opening, an orifice near the distal end of the supply lumen, the orifice being configured to allow injection of pressurized fluid into the aspiration lumen at or near the distal end of the aspiration lumen when the pressurized fluid is pumped through the supply lumen, and a tissue encroachment prevention assembly for preventing the blood vessel from being drawn into the aspiration lumen and into close proximity to the orifice of the supply lumen during aspiration, the tissue encroachment prevention assembly comprising at least one of a cage-like structure.

Embodiment 20. The aspiration catheter of embodiment 19, said cage-like structure comprising a plurality of tines extending from a point at or near the opening at the distal end of the elongate shaft, the plurality of tines each having distal ends that are attached to a spherical ball and proximal ends that extend from the distal end of the elongate shaft, wherein the plurality of tines attached to the spherical ball prevent vessel tissue from being drawn into the aspiration lumen and into close proximity to the orifice of the supply lumen.

Embodiment 21. The aspiration catheter of any of embodiments 19-20, wherein at least 3 tines extend from the aspiration lumen.

Embodiment 22. The aspiration catheter of any of embodiments 19-21, wherein the tines contain a spring element that allows the tines to bend.

Embodiment 23. The aspiration catheter of any of embodiments 19-22, further comprising a radiopaque ring encircling the catheter near the distal end of the aspiration catheter.

Embodiment 24. The aspiration catheter of any of embodiments 19-23, wherein the plurality of tines extend from a base having holes dispersed throughout.

Embodiment 25. The aspiration catheter of any of embodiments 19-24, wherein a width of the tines is tapered towards the spherical ball.

Embodiment 26. The aspiration catheter of any of embodiments 19-25, wherein the tines are laser welded or soldered directly to a surface of the spherical ball.

Embodiment 27. The aspiration catheter of any of embodiments 19-26, wherein the spherical ball comprises a blind hole to receive the tines.

Embodiment 28. The aspiration catheter of any of embodiments 19-27, wherein the spherical ball is made of a radiopaque material.

Embodiment 29. The aspiration catheter of any of embodiments 19-28, further comprising a mesh configuration at the proximal ends of the tines.

Embodiment 30. The aspiration catheter of any of embodiments 19-29, further comprising one or more supply lumens, each having an orifice configured to allow injection of pressurized fluid into the aspiration lumen at or near the distal end of the aspiration lumen when the pressurized fluid is pumped through the supply lumen.

Embodiment 31. The aspiration catheter of any of embodiments 19-30, wherein the elongate shaft is a laser cut hypotube laminated by a polymer.

Embodiment 32. The aspiration catheter of any of embodiments 19-31, wherein the elongate shaft is a multi-lumen extruded polymer shaft.

Embodiment 33. The aspiration catheter of any of embodiments 19-32, further comprising a radiopaque ring near the distal end of the aspiration catheter.

Embodiment 34. A system for aspirating thrombus, comprising an aspiration catheter comprising an elongate shaft configured for placement within a blood vessel of a subject, a supply lumen and an aspiration lumen each extending along an interior of the elongate shaft, and an orifice at or near a distal end of the supply lumen, the orifice being configured to allow injection of pressurized fluid into the aspiration lumen at or near the distal end of the aspiration lumen when the pressurized fluid is caused or allowed to flow through the supply lumen, a tubing set comprising a first conduit having a distal end configured to couple to the aspiration lumen of the aspiration catheter and a proximal end configured to couple to a vacuum source, and a second conduit having a distal end configured to couple to the supply lumen of the aspiration catheter and a proximal end configured to couple to a first fluid source, a pressurization element configured to couple to the tubing set and further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen and a means for preventing the blood vessel from being drawn into the aspiration lumen and into close proximity to the orifice of the supply lumen during aspiration.

Embodiment 35. The system for aspirating thrombus of embodiment 34, wherein the means for preventing the blood vessel from being drawn into the aspiration lumen and into close proximity to the orifice of the supply lumen during aspiration comprises a cage-like structure formed from a plurality of tines which extend from the distal end of the aspiration catheter and which each have distal ends that are attached to a spherical ball.

Embodiment 36. A system for aspirating thrombus, comprising an aspiration catheter comprising an elongate shaft configured for placement within a blood vessel of a subject, a supply lumen and an aspiration lumen each extending along an interior of the elongate shaft, and an orifice at or near a distal end of the supply lumen, the orifice being configured to allow injection of pressurized fluid into the aspiration lumen at or near the distal end of the aspiration lumen when the pressurized fluid is caused or allowed to flow through the supply lumen, a tubing set comprising a first conduit having a distal end configured to couple to the aspiration lumen of the aspiration catheter and a proximal end configured to couple to a vacuum source, and a second conduit having a distal end configured to couple to the supply lumen of the aspiration catheter and a proximal end configured to couple to a first fluid source, a pressurization element configured to couple to the tubing set and further configured to pressurize fluid from the first fluid source or allow pressurized fluid from the first fluid source to be transferred to the supply lumen, such that the pressurized fluid is capable of flowing through the supply lumen from the proximal end of the supply lumen to the orifice at or near the distal end of the supply lumen, and a tissue encroachment prevention assembly for preventing the blood vessel from being drawn into the aspiration lumen and into close proximity to the orifice of the supply lumen during aspiration.

Embodiment 37. The system for aspirating thrombus of embodiment 36, wherein the tissue encroachment prevention assembly for preventing the blood vessel from being drawn into the aspiration lumen and into close proximity to the orifice of the supply lumen during aspiration comprises a cage-like structure formed from a plurality of tines which extend from the distal end of the aspiration catheter and which each have distal ends that are attached to a spherical ball.

Embodiment 38. An aspiration catheter comprising an elongate shaft configured for placement within a blood vessel, a supply lumen and an aspiration lumen each extending along an interior of the shaft, coextensive with the shaft, the supply lumen having a proximal end and a distal end, and the aspiration lumen having a proximal end and a distal opening, an orifice at or near the distal end of the supply lumen, the orifice configured to allow injection of pressurized fluid into the aspiration lumen at or near the distal end of the aspiration lumen when the pressurized fluid is pumped through the supply lumen, and a means for preventing the blood vessel from being drawn into said aspiration lumen and coming into close proximity to the orifice of the supply lumen during aspiration.

Embodiment 39. An aspiration catheter comprising: an elongate shaft configured for placement within a blood vessel, the elongate shaft having an aspiration lumen extending therethrough, the aspiration lumen having an opening at a distal end of the elongate shaft; a supply lumen extending along the shaft, the supply lumen having a distal end and an orifice at or near the distal end, the orifice being configured to allow injection of pressurized fluid into the aspiration lumen at or near a distal end of the aspiration lumen when the pressurized fluid is delivered through the supply lumen; and a tissue encroachment prevention assembly for preventing the blood vessel from being drawn into the aspiration lumen and coming into close proximity to the orifice of the supply lumen during aspiration, the tissue encroachment prevention assembly comprising one or more atraumatic members that expend distally from the elongate shaft and across the opening to the aspiration lumen.

Embodiment 40. The aspiration catheter of Embodiment 39, wherein at least one of the one or more atraumatic members comprises a wire having a first end and a second end that are connected to or otherwise associated with the distal end of the shaft.

Embodiment 41. The aspiration catheter of Embodiment 40, wherein at least a portion of the atraumatic member between the first and second ends thereof has and arcuate shape.

Embodiment 42. The aspiration catheter of Embodiment 40, wherein the first end and the second end are both fixedly attached to the shaft.

Embodiment 43. The aspiration catheter of Embodiment 40, wherein the first end is fixedly attached to the shaft and the second end is movable relative to the shaft such that the atraumatic member is selectively movable between a retracted state and a deployed state.

Embodiment 44. The aspiration catheter of Embodiment 43, wherein a portion of the atraumatic member is configured to move from a first shape when in the retracted state to a second shape when in the deployed state.

Embodiment 45. The aspiration catheter of Embodiment 43, wherein the second end is disposed within a tissue encroachment prevention assembly lumen within the shaft when in the retracted state.

Embodiment 46. The aspiration catheter of Embodiment 45, wherein the tissue encroachment prevention assembly further comprises an elongate member associated with the second end of the atraumatic member.

Embodiment 47. The aspiration catheter of Embodiment 46, wherein the elongate member is movably disposed within the tissue encroachment prevention assembly lumen.

Embodiment 48. The aspiration catheter of Embodiment 39, wherein at least one of the one or more atraumatic members comprises a radiopaque material.

Embodiment 49. The aspiration catheter of Embodiment 40, wherein at least one of the one or more atraumatic members comprises a length and a width, the length being a distance between the distal end of the shaft and a distal-most portion of the at least one atraumatic member, and the width being a distance between the first and second ends of the wire.

Embodiment 50. The aspiration catheter of Embodiment 49, wherein the length is greater than the width.

Embodiment 51. The aspiration catheter of Embodiment 49, wherein the length less than the width.

Embodiment 52. The aspiration catheter of Embodiment 49, wherein the length is equal to the width.

1. An aspiration catheter comprising:
an elongate shaft configured for placement within a blood vessel;
a supply lumen and an aspiration lumen each extending along the shaft, the supply lumen having a proximal end and a distal end, and the aspiration lumen having a proximal end and a distal opening;
an orifice at or near the distal end of the supply lumen, the orifice being configured to allow injection of pressurized fluid into the aspiration lumen at or near the distal end of the aspiration lumen when the pressurized fluid is delivered through the supply lumen; and
a tissue encroachment prevention assembly for preventing the blood vessel from being drawn into said aspiration lumen and coming into close proximity to the orifice of the supply lumen during aspiration.

2. The aspiration catheter of claim 1, wherein said tissue encroachment prevention assembly comprises:
a plurality of tines extending from a point at or near the opening at the distal end of the elongate shaft, the plurality of tines each having distal ends that are attached to a spherical ball and proximal ends that extend from the distal end of the elongate shaft;
wherein the plurality of tines attached to the spherical ball are configured to form a cage-like structure that prevents vessel tissue from being drawn into the aspiration lumen and into close proximity to the orifice of the supply lumen.

3. The aspiration catheter of claim 2, wherein at least three tines extend from the aspiration lumen.

4. The aspiration catheter of claim 2, wherein the tines contain a spring element that allows the tines to bend.

5. The aspiration catheter of claim 2, wherein a width of the tines is tapered towards the spherical ball.

6. The aspiration catheter of claim 2, wherein the spherical ball is made of a radiopaque material.

7. The aspiration catheter of claim 2, further comprising a mesh configuration at the proximal ends of the tines.

8. An aspiration catheter comprising:
an elongate shaft configured for placement within a blood vessel, the elongate shaft having a distal end with an opening at the distal end;
a supply lumen and an aspiration lumen each extending along the shaft, the supply lumen having a proximal end and a distal end, and the aspiration lumen having a proximal end and a distal opening;
an orifice near the distal end of the supply lumen, the orifice being configured to allow injection of pressurized fluid into the aspiration lumen at or near the distal end of the aspiration lumen when the pressurized fluid is delivered through the supply lumen; and
a means for preventing the blood vessel from being drawn into the aspiration lumen and into close proximity to the orifice of the supply lumen during aspiration.

9. The aspiration catheter of claim 8, wherein the means for preventing the blood vessel from being drawn into the aspiration lumen comprises a cage-like structure extending distally from a point at or near the opening at the distal end of the elongate shaft.

10. The aspiration catheter of claim 9, wherein the cage-like structure comprises a plurality of tines, the plurality of tines each having distal ends that are attached to a spherical ball to form the cage-like structure and proximal ends that extend from the distal end of the elongate shaft.

11. An aspiration catheter comprising:
an elongate shaft configured for placement within a blood vessel, the elongate shaft having a distal end with an opening at the distal end;
a supply lumen and an aspiration lumen each extending along an interior of the shaft, coextensive with the shaft, the supply lumen having a proximal end and a distal end, and the aspiration lumen having a proximal end and a distal opening;
an orifice near the distal end of the supply lumen, the orifice being configured to allow injection of pressurized fluid into the aspiration lumen at or near the distal end of the aspiration lumen when the pressurized fluid is pumped through the supply lumen; and
a tissue encroachment prevention assembly for preventing the blood vessel from being drawn into the aspiration lumen and into close proximity to the orifice of the supply lumen during aspiration, the tissue encroachment prevention assembly comprising at least one of a cage-like structure.

12. The aspiration catheter of claim 11, said cage-like structure comprising a plurality of tines extending from a point at or near the opening at the distal end of the elongate shaft, the plurality of tines each having distal ends that are attached to a spherical ball and proximal ends that extend from the distal end of the elongate shaft, wherein the plurality of tines attached to the spherical ball prevent vessel tissue from being drawn into the aspiration lumen and into close proximity to the orifice of the supply lumen.

13. The aspiration catheter of claim 12, wherein at least three tines extend from the aspiration lumen.

14. The aspiration catheter of claim 12, wherein the tines contain a spring element that allows the tines to bend.

15. The aspiration catheter of claim 12, wherein the plurality of tines extend from a base having holes dispersed throughout.

16. The aspiration catheter of claim 12, wherein a width of the tines is tapered towards the spherical ball.

17. The aspiration catheter of claim 12, wherein the tines are laser welded or soldered directly to a surface of the spherical ball.

18. The aspiration catheter of claim 12, wherein the spherical ball comprises a blind hole to receive the tines.

19. The aspiration catheter of claim 12, wherein the spherical ball is made of a radiopaque material.

20. The aspiration catheter of claim 12, further comprising a mesh configuration at the proximal ends of the tines.
